(19) United States
(12) Patent Application Publication
(10) Pub. No.: US 2007/0248599 A1
(43) Pub. Date: Oct. 25, 2007

(54) TREATMENT OF ALZHEIMER'S DISEASE WITH INHIBITORS OF APOE BINDING TO APOE RECEPTOR

(76) Inventor: Jordan Tang, Edmond, OK (US)

Correspondence Address: FULBRIGHT & JAWORSKI L.L.P., 600 CONGRESS AVE., SUITE 2400, AUSTIN, TX, 78701, US

(21) Appl. No.: 11/676,042

(22) Filed: Feb. 16, 2007

Related U.S. Application Data
(60) Provisional application No. 60/775,477, filed on Feb. 21, 2006.

(51) Int. Cl. A61K 39/395; A61K 31/7052; A61K 48/00

(52) U.S. Cl. 424/133.1; 424/130.1; 514/2; 514/44

(57) ABSTRACT

Alzheimer's disease (AD) is characterized by the accumulation of amyloid-β peptide (Aβ) in the brain. Aβ is derived from amyloid precursor protein (APP) by β- and γ-secretases. Apolipoprotein E receptor 2 (ApoER2) is a cell-surface receptor for apolipoprotein E. This study shows that ApoER2 interacts with X11α/β proteins and that APP forms association with ApoER2 in the presence of X11s. Significantly, ApoE stimulates the production of Aβ, and ApoE4 produced more Aβ than ApoE2 or ApoE3, correlating with previous studies showing that individuals with the ApoE4 polymorphism were more prone to development of AD. Thus, ApoE binding to ApoER2 on cell surface stimulates the generation of Aβ from APP. Antagonists that interfere with the ApoE-ApoER2 interaction are proposed for the treatment of AD.

The present application claims benefit of priority to U.S. Provisional Application Ser. No. 60/775,477, filed Feb. 21, 2006, the entire contents of which are hereby incorporated by reference.

The United States Government owns rights in the invention pursuant to funding from the National Institutes of Health under Grant No. AG-18933.

### BACKGROUND OF THE INVENTION

I. Field of the Invention

The present invention relates generally to the fields of neuropathology and molecular biology. More particularly, it concerns the use of agents that inhibit the interaction of ApoE, including ApoE4, with the ApoE receptor, such as ApoER2.

II. Description of Related Art

Neurodegenerative diseases are generally characterized by the loss of neurons from one or more regions of the central nervous system. They are complex in both origin and progression, and have proved to be some of the most difficult types of disease to treat. In fact, for some neurodegenerative diseases, there are no drugs available that provide significant therapeutic benefit. The difficulty in providing therapy is all the more tragic given the devastating effects these diseases have on their victims.

One type of neurodegenerative disease is Alzheimer's disease (AD), the most common form of dementia among older people. Scientists believe that up to 4 million Americans suffer from AD. The disease usually begins after age 60, and risk goes up with age. While younger people also may get AD, it is much less common. About 3 percent of men and women ages 65 to 74 have AD, and nearly half of those age 85 and older may have the disease. While the subject of intensive research, the precise causes of AD are still unknown, and there is no cure.

AD attacks parts of the brain that control thought, memory, and language. It was named after Dr. Alois Alzheimer, a German doctor. In 1906, Dr. Alzheimer noticed changes in the brain tissue of a woman who had died of an unusual mental illness. He found abnormal clumps, now called amyloid “plaques,” and tangled bundles of fibers, now called neurofibrillary “tangles.” Today, these plaques and tangles in the brain are considered hallmarks of AD.

The production, aggregation, and accumulation of amyloid β-protein (Aβ), the major constituent of the amyloid plaque, in the brain are initial steps in the pathogenesis of AD. Aβ is generated by the intracellular processing of amyloid β precursor protein (APP, see FIG. 1) (Selkoe, 2001), a type I membrane protein (Kang et al., 1987), by proteases β-secretase (memapsin 2 or BACE1) and γ-secretase. The cytoplasmic domain of APP (APPcyt), through its interactions with cytoplasmic proteins, plays an important role in the regulation of APP metabolism and Aβ production (King and Turner, 2004).

Among the proteins that interact with APPcyt are X11 family proteins, X11α/β/γ, (also known as X11, X11-like (X11L) and X11-like 2 (X11L2)). FIG. 1 shows a diagram of the domains in X11 proteins. While X11γ is ubiquitously distributed in different tissues, X11α and β are expressed only in the brain (Borg et al., 1999; Hase et al., 2002). Each of the family proteins was reported to stabilize intracellular APP and/or suppress Aβ production (King and Turner, 2004).

ApoE receptor 2 (ApoER2), a member of the LDL receptor family, is predominantly expressed in the brain (Kim et al., 2001). ApoER2 is a receptor for apolipoprotein E (ApoE) and Reelin, a signaling protein that regulates neuronal migration during brain development (D'Arcangelo, 1999). The binding of ApoE to ApoER2 results in the transportation of the complex into cells, as part of a process by which lipid is transported into cells, ApoE being one of the carrier proteins.

There are three structurally different ApoE's in man: ApoE2, ApoE3 and ApoE4. The ApoE polymorphism is linked to AD as people with ApoE4 have higher incidence of the disease (Beffert et al, 2004). The physiological mechanism for this risk is not clear. Several lines of indirect evidence suggest that ApoER2 may play a role in AD. It has been shown (Motoi, 2004) that ApoER2 is present in amyloid plaques of AD patients, implying a possible involvement of ApoER2 in plaque formation. ApoER2 is also involved in the maintenance of efficient synaptic plasticity (Petit-Turcotte et al., 2005). Moreover, both APP and ApoER2 bind to Dab1 and JIP1 (King and Turner, 2004), suggesting related cellular functions. However, the precise role played by ApoER2 and ApoE4 in AD remains unclear.

### SUMMARY OF THE INVENTION

Thus, in accordance with the present invention, there is provided a method of reducing Aβ production in a neuronal cell expressing an ApoER2 receptor comprising providing to said cell an agent that inhibits the binding of ApoE to ApoER2. In another embodiment, there is provided a method of inhibiting Aβ plaque formation in neurons of a subject comprising providing to subject an agent that inhibits the binding of ApoE to ApoER2. In yet another embodiment, there is provided a method of blocking the progression of one or more symptoms of Alzheimer's Disease in a subject comprising providing to subject an agent that inhibits the binding of ApoE to ApoER2. And it still another embodiment, there is provided a method of delaying the onset of Alzheimer's Disease in a subject comprising providing to subject an agent that inhibits the binding of ApoE to ApoER2.

The agent may preferentially inhibit the interaction of ApoE4 binding to ApoER2. The agent may be a soluble form of ApoER2 or an ApoER2 peptide, an ApoE peptide, such as an ApoE4 peptide, and in particular an ApoE4 peptide comprising position 112 of the native ApoE4 protein. The agent may also be an antibody or antibody fragment that binds to ApoER2 or ApoE4, such as a single chain antibody or a humanized antibody. The agent may reduce the expression of ApoER2 in said neuronal cell, for example, such agents including a small molecule, an ApoER2 antisense molecule, an ApoER2 siRNA, or an ApoER2 ribozyme. The agent may instead reduce the expression of ApoE4 in a cell expressing ApoE4, again, being a small molecule, an ApoE4 antisense molecule, an ApoE4 siRNA, or an ApoE4 ribozyme.

The agent may be delivered in a lipid vehicle, e.g., a liposome or a nanoparticle The delivery may comprise contacting a cell with an expression construct encoding said agent under the control of a promoter. The expression construct may be a viral expression construct, such as a neurotrophic virus, including a retrovirus, a lentivirus, or a herpesvirus. The neuronal cell may be a human neuronal cell, such as one in a living subject. The subject may suffer from Alzheimer's Disease or not, or may have a pre-existing Aβ plaque.

Also provided are pharmaceutical compositions comprising an agent that preferentially inhibits the interaction of ApoE4 binding to ApoER2. The composition may comprise an agent that is a soluble form of ApoER2, an ApoER2 peptide, an ApoE peptide, such as an ApoE4 peptide, in particular an ApoE4 peptide comprising position 112 of the native ApoE4 protein, an antibody or antibody fragment that binds to ApoER2 or ApoE4, or a single chain antibody or a humanized antibody. The composition may also comprise an agent that reduces the expression of ApoER2 in said neuronal cell, for example, a small molecule, an ApoER2 antisense molecule, an ApoER2 siRNA, or an ApoER2 ribozyme. The composition may also comprise an agent that reduces the expression of ApoE4 in a cell expressing ApoE4, again, a small molecule, an ApoE4 antisense molecule, an ApoE4 siRNA, or an ApoE4 ribozyme.

In still another embodiment, there is provided a method of reducing Aβ production in a neuronal cell expressing an ApoER2 receptor comprising providing to said cell an agent that inhibits the binding of X11α/β to ApoER2. The agent may be a dominant-negative form of X11α/β, such as PTB domain peptide, including one having SEQ ID NO:9, SEQ ID NO:10 or SEQ ID NO:12, or an inhibitory fragment thereof. The agent may also be a an antibody or antibody fragment that binds to ApoER2 or X11α/β, such as a antibody is a single chain antibody or a humanized antibody. The agent may be a peptidomimetic of X11α/β ApoE. The agent may also be a small molecule that minics the conformations of either ApoER2 or ApoE (including all three polymorphic forms), resulting in interference of ApoE binding to ApoER2. The agent may be delivered in a lipid vehicle, such as a liposome. The neuronal cell may be a human neuronal cell, and may be located in a living subject. The living subject may or may not suffer from Alzheimer's Disease, and may or may not have pre-existing Aβ plaques.

In other embodiments, there are provided: a method of inhibiting Aβ plaque formation in neurons of a subject comprising providing to said cell an agent that inhibits the binding of X11α/β to ApoER2; a method of blocking the progression of one or more symptoms of Alzheimer's Disease in a subject comprising providing to said cell an agent that inhibits the binding of X11α/β to ApoER2; and a method of delaying the onset of Alzheimer's Disease in a subject comprising providing to said cell an agent that inhibits the binding of X11α/β to ApoER2.

In still additional embodiments, there are provided: a dominant negative X11α/β, such as one lacking the PDZ domain; a peptide comprising a phosphotyrosine/tyrosine binding (PTB) domain of X11α/β, said peptide being 10 to 50 residues in length, such as one comprising a 10 to 50 residue contiguous segment of SEQ ID NO:9, SEQ ID NO:10 or SEQ ID NO:12 or residues 413 to 421 of SEQ ID NO:8; an antibody that binds to the phosphotyrosine/tyrosine binding (PTB) domain of X11α/β, such as a single chain antibody or a humanized antibody; or an antisera, antibodies of which bind to the phosphotyrosine/tyrosine binding (PTB) domain of X11α/β.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention.

FIGS. 1A-C—(FIG. 1A) Diagram of human Swedish APP695 structure and intracellular processing of Aβ. (FIG. 1B) Representation of domain structure of human X11α/β mammalian structure. (FIG. 1C) Diagram of the organization of murine wild-type ApoER2 construct which containing 59 amino acid insertion in intracellular domain.

FIGS. 2A-B—Co-immunoprecipitation of ApoER2 with X11α/β. Cell lysates of HEK293 cells transiently overexpressing ApoER2 and X11α/β were precipitated with anti-X11α/anti-myc for X11β or with control mouse IgG. The immunoprecipitated proteins were separated on SDS-PAGE. The pellets were blotted with anti-V5 for detecting ApoER2 and were blotted with corresponding antibodies for X11α/β after stripping. ApoER2 was found can co-immunoprecipitate with anti-X11α/β but not the control IgG.

FIG. 3—APP co-immunoprecipitate with ApoER2 together with X11α/β. HEK 293 cell lysates expressing APPsw with ApoER2 or APP, ApoER2 together with X11α/β were precipitated with anti APP 5228 or control mouse IgG. The pellets were separated by SDS-PAGE and detected by Western Blotting using anti-V5 for ApoER2. No ApoER2 was detected in lanes 1 and 2 when only APP and ApoER2 were expressed in the cells. ApoER2 was found can co-immunoprecipitate with APP when coexpressed with X11α (lane 3, 4) or X11β (lane 5, 6).

FIG. 4—ApoE stimulatory effect on Aβ40 secretion depends on interaction with ApoER2. In N2a APPsw cells, apoE, apoE4 (5 μg/mL) were added into OPTIM medium. The experimental group was transfected with murine ApoER2 plasmids, same amount of pcDNA6.1 plasmids used as control. After overnight treatment, same amount of conditioned medium was collected for ELISA to detect Aβ40. Each experiment was repeated at least three times. The P values between experimental group and control are <0.05. Overexpressing ApoER2 compared with control showed obvious increase of stimulation on Aβ40 secretion.

FIGS. 5A-E—ApoE increases Aβ production in neuroblastoma N2a-APPsw cells. (FIG. 5A) Human VLDL (10 μg/mL) increased Aβ production in N2a-APPsw cells. HDL and LDL (same concentration) had no effect. Aβ in the medium was analyzed after 24 h of culture. Inset: Western blots of ApoE in three lipoprotein fractions. (FIG. 5B) Purified human ApoE, recombinant ApoE2, ApoE3 and ApoE4 (each at 5 μg/mL) increased Aβ production in N2a-APPsw cells. Experiment conditions same as in (a). *, p<0.05; **, p<0.01. (FIG. 5C) Correlation of ApoE4 content of human VLDL with Aβ₄₀ and Aβ₄₂ production in N2a-APPsw cells. VLDL samples purified from individuals were analyzed for ApoE4 and total ApoE contents and measured with ELISA for their effect on Aβ₄₀ and Aβ₄₂ production in N2a-APPsw cells. (FIG. 5D) Transfect of ApoER2 into N2a-APPsw cells increased ApoE or VLDL induced increase of Aβ production (black columns) over cells transfected with blank vectors (gray columns). The experimental conditions were the same as above. (FIG. 5E) ApoER2 siRNA knock down abolished ApoE or ApoE4 induced increase of Aβ production (left panel). Western blots (right panel) show that the endogenous ApoER2 band was greatly reduced in ApoER2 siRNA knock down while the control β-actin band was not significantly changed.

FIGS. 6A-D—ApoE or ApoE4 induced the internalization of ApoER2, APP and β-secretase from the cell surface. (FIG. 6A) ApoE reduced cell surface ApoER2, APP and α-secretase. N2a-APPsw cells transfected with ApoER2 and β-secretase were incubated with ApoE or ApoE4 for 2 h. Cell surface proteins were biotinylated, retrieved with avidin-argarose gel, and subjected to Western blots for ApoER2, APP and β-secretase (upper panel, right). Quantitation of the bands was done by scanning densitometer and displayed here as relative intensity with the controls (white columns) set as 1.0 (lower panel, average of 2 experiments). Parallel experiments were carried out without biotinylation from which the lysates were subjected to Western blots to estimate the total amount of three proteins in the cells (lower panel). (FIG. 6B) ApoE increased intracellular ApoER2, APP and β-secretase. Cells were prepared as in FIG. 6A, then the cell surface proteins labeled with a cleavable biotinylation reagent at 4° C. The internalization was affected at 37° C. for 15 min. After stripping the biotinyl group on cell surface, the cells were lysed and the biotinylated proteins were retrieved for Western blots as above. The displayed panels are the same as in, FIG. 6A except that in the lower panel, the amounts of ApoE (black column) is set as 1.0. (FIG. 6C) Western blot of intracellular APP fragment C99 from N2a-APPsw cells in the absence and presence of ApoE. The samples were those described in FIG. 6B, blotted with antibody MAB1560. (FIG. 6D) Western blot of soluble APP ectodomain generated by ∝-secretase, s∝-APP, from N2a-APPsw cells in the absence and presence of ApoE. The condition medium from FIG. 6B was immuno-precipitated with MAB1560 and Western blotted with the same antibody.

FIGS. 7A-E—Binding of X11α/β with ApoER2. (FIG. 7A) Diagrams of some functional domains of APP, ApoER2 and X11. In APP, a dot denotes the location of the YENPTY motif which involves in binding of several adaptor proteins including X11α/β. KPI is a Kunitz protease inhibitor domain. In ApoER2, the alternative spliced 59-residue insertion is shown in blue. (FIG. 7B) ApoER2 is recovered in immunoprecipitation of X11α/β. Lysates of HEK293 cells transiently expressing ApoER2 (with V5 tag) and X11α or X11β (with myc tag) were immunoprecipitated separately with anti-X11α, anti-myc (for X11β) or control IgG, and visualized in Western blots with anti-V5, anti-X11α or anti-myc. ApoER2 band was present in both immunoprecipitation for X11α and X11β. (FIG. 7C) LRP (LDL receptor related protein) did not co-immunoprecipitate with X11α. Experimental conditions same as in FIG. 7A. (FIG. 7D) Presence of X11α/β in ApoER2 pull-down experiments. Lysates of cells expressing ApoER2 (with both V5 and hexahistidine tags) and X11α/β were subjected to binding by Ni-affinity gel to retrieve ApoER2. Western blots showed that both X11α (upper panel) and X11β (lower panel) were recovered. (FIG. 7E) ApoER2 and X11α/β are immunoprecipitated with APP. Lysates of HEK293 cells transiently expressing APP, ApoER2 and X11α or X11β were immunoprecipitated with anti-APP antibody 5228 and visualized by Western blots. ApoER2 was present when X11α/β was expressed (lanes 3 & 5) but was absent when X11 was not expressed (lane 1).

FIGS. 8A-D—Domains and motif involved in ApoER2/X11α/β interaction. (FIG. 8A) PTB domain of X11α binds ApoER2. Lysates from cells expressing ApoER2 and GST (glutathione-5-transferase) fusions of PTB or PDZ domains of X11α were subjected to GST pull down using a glutathione affinity column. Western blots showed that ApoER2 was associated with PTB domain (third lane) but not PDZ domains (second lane). (FIG. 8B) X11α pull down by GST (white bar) fusion constructs containing different regions of ApoER2 intracellular regions (gray bar). GST fusion to APP cytosolic domain (slash bar) was the positive control. Constructs containing the 59-residue insertion (check bar) in GST-ApoC1 & GST-ApoC3 were able to pull down X11α from cell lysate, as shown in Western blot. (FIG. 8C) GST-PTB (X11α) pull down of ApoER2 mutants as shown by Western blot (upper panel). The lower panel shows a diagram of mutation positions. Deletion of the NPTY (SEQ ID NO:5) motif (mutants 1 & 2) or mutation of a PXXP (SEQ ID NO:6) motif (mutant 3 & 4) did not affect the pull down. Only the deletion of the YDRPLW (SEQ ID NO:7) motif abolished the pull down. GST was the negative control in pull down and Western blot. (h) Reversed pull down from (FIG. 8D) where wide-type ApoER2 pulled down X11α (lane 2) but Mutant 5 (ApoER2M5) did not (lane 4).

FIGS. 9A-C—Involvement of X11α/β in APP and ApoER2 internalization and ApoE induced Aβ, production. (FIG. 9A) Intercellular localization of APP with ApoER2 (FIGS. 9A-C) and ApoER2 with X11α/β (FIGS. 9D-I). Arrows in the merged images point to subcellular compartments consistent with endosomes. Scale bar represents 10 μm. (FIG. 9B) Expression of PTB domain of X11α/β abolished ApoE induced Aβ production. (FIG. 9C) Deletion of X11α/β-binding motif YDRPLW (SEQ ID NO:7) in ApoER2 (Mut4, mutant 4 in FIG. 8C) abolished ApoE induced Aβ production.

FIGS. 10A-B. Absence of Reelin and a reelin effect in ApoE triggered Aβ increase in N2a-APPsw cells. (FIG. 10A) Western blots for Reelin in cell lysate and culture medium of N2a-APPsw cells (left two lanes) and N2a-APPsw cells transfected with a Reelin expression vector. (FIG. 10B) Aβ40 production in N2a-APPsw cells in the presence (right column) and absence of anti-Reelin antibody (left and center columns). Values were averaged from 3 determinations.

FIG. 11—Schematic presentation of ApoE triggered Aβ production. At cell surface, ApoER2 (blue) and APP (red) each bind a molecule of X11α/β (green) to form a complex with the possible involvement of other cytosolic proteins (represented by a beige oval) during the recruitment and packaging of endocytic vescicles. β-Secretase is associated with this complex by virtue of its recognition of APP, where the protease is inactive at pH 7. In the presence of ApoE-containing lipid particles, ApoE binds ApoER2 to trigger endocytosis of the complex with β-secretase to intracellular compartments where, at pH 4.5, APP is cleaved by β-secretase and γ-secretase to generate Aβ. In this mechanism, ApoE triggers the production of Aβ, where ApoE4 generates more Aβ than do ApoE2 and ApoE3, possibly as a result of a stronger association with ApoER2. This mechanism also links Aβ production to lipid uptake which may be associated with neuronal activities.

### DESCRIPTION OF ILLUSTRATIVE EMBODIMENTS

Neurodegenerative diseases are particularly devastating in that they progressively incapacitate their victims. Remarkably, though much progress has been made in recent years, there remain relatively few drugs that are useful in the treatment of neurodegenerative diseases, and almost none that are effective for a high percentage of patients. Thus, there is an urgent need for new and improved drugs and methods of therapy for these conditions, which includes Alzheimer's Disease, a condition that has devastating effects on cognitive function and overall mental health costing billions of dollars in healthcare for the elderly.

### I. The Present Invention

The present inventors now show that ApoER2 mediates the increase of Aβ40 production by ApoE, especially ApoE4, in neuronal cells. The explanation of this relationship is as follows. In the presence of X11, APP is associated with ApoER2. The binding of ApoE to ApoER2 results in the endocytosis of the ApoE-ApoER2 complex from cell surface into endosomes. In the presence of X11, APP becomes endocytosed with this complex. Since endosomes are the main site for APP cleavages to form Aβ, this process can be expected to increase Aβ production and secretion. ApoE4 apparently is more effective in carrying out this process that the other ApoE's.

This discovery also predicts that small molecular antagonists that interfere with the binding of ApoER2 to ApoE, especially ApoE4, will reduce the production of Aβ. These antagonist molecules can be used to treat Alzheimer's disease or to lower the risk that this disease will develop. Peptide sequences around the known polymorphic sites in ApoE, including ApoE4, are potential agonists, as are portions of the PTB binding domains of X11α/β/γ. Interaction of ApoER2 and ApoE can be monitored with various methods, such as using surface plasmon in a BIAcore instrument. Alternatively, antibodies of ApoER2 may be used to interfere with ApoE binding and to lower Aβ production. Other drugs may be designed to lower the number of ApoER2 molecules in neurons by influence the biosynthesis, distribution or degradation of ApoER2.

### II. Alzheimer's Disease

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

### III. ApoE and ApoER2

A. ApoE

Apolipoproteins are carrier proteins that combine with lipids to form lipoprotein particles, which have hydrophobic lipids at the core and hydrophilic side chains made of amino acids. There are nine different apolipoproteins that found in humans. One of these, apolipoprotein E (ApoE) has many functions in the body. When synthesized by the liver as part of VLDL, it transports triglycerides to liver tissue. It plays a role in HDL to help distribute cholesterol among cells. It is also incorporated into intestinally synthesized chylomicrons and transports dietary triglycerides and cholesterol. Finally, it mediate the binding to the LDL, which begins the process of cellular uptake of lipoproteins for in intracellular cholesterol metabolism.

ApoE is a 299 amino acid protein with a predicted molecular weight of approximately 34,000 (accession No. P02649 (SEQ ID NO:1), incorporated by reference). The gene for ApoE is found on chromosome 19 and is 3.7 kb in length. The transcript is 1163 base pairs long, but undergoes posttranslational processing. ApoE is synthesized primarily in the liver, but the brain also produces a large amount of ApoE. ApoE is also synthesized in the spleen, lungs, adrenals, ovaries, kidneys, muscle cells, and in macrophages. ApoE is normally present in plasma at 5 mg/dl, and it associates with chylomicrons, VLDL, and HDL.

The structure of ApoE can be divided into three sections. The amino-terminal end, up to residue 165, is highly ordered. The next 35 residues make up a random structure. The carboxyl-terminal portion is also highly ordered. The area of the protein with the strongest lipid binding is found at residues 202-209. Five arginine and three lysine residues between residues 140 and 160 are essential for binding to the LDL (low-density lipoprotein) lipid receptor, which is believed due to the ionic interactions between the basic residues of the ApoE and the acidic residues (from aspartic and glutamic acids) of the lipid receptor.

There are three different isoforms of apolipoprotein E: ApoE2, ApoE3, and ApoE4. ApoE3 is the reference sequence to which others are compared. ApoE2 differs in that a cysteine is substituted for arginine of the E2 form at residue 158. ApoE2 is associated with Type III Hyperproteinemia; in fact, ApoE2 shows less than 2% of the normal receptor binding activity. ApoE4 has an arginine substituted for cysteine at residue 112. This residue is outside of the strongest lipid binding area and the substitution does not affect the lipid binding ability. ApoE4 still has 100% of normal receptor binding activity.

B. ApoER2

The cDNA for the apolipoprotein E receptor 2 (ApoER2) shows strong homology with the LDL-R and the VLDL receptor. The accession no. for ApoER2 is Q14114 (SEQ ID NO:2), for LDL-R is NP-000518 (SEQ ID NO:3); and for VLDL-R is AAB31735 (SEQ ID NO:4), all of which are incorporated by reference. In common with other LDL-R family members, ApoER2 contains the cytoplasmic sequence NPxY required for internalization of the LDL-R via clathrin-coated pits. However, this motif is also a ligand for the phosphotyrosine binding and PDZ domains of signaling molecules, some of which can bind to ApoER2. The cellular role of apoER2 is unlikely to involve lipoprotein uptake and degradation, a conclusion recently substantiated by direct analysis of the endocytic function of cytoplasmic ApoER2. Sun & Soutar (1999). This implies an alternative function for the NPxY motif in apoER2. Indeed, there is increasing evidence that ApoER2 is a signal transducer molecule, regulating neuronal migration during brain development, and perhaps moderating platelet aggregation in the vasculature. Trommsdorff et al. (1999); Riddell et al. (1999). Signaling appears to require interaction between the cytoplasmic domain of apoER2 and adapter molecules such as Dab1, JIP 1 and JIP 2, and PSD-95. Trommsdorff et al. (1999); Gotthardt et al. (2000); Stockinger et al. (2000).

C. X11α/β/γ

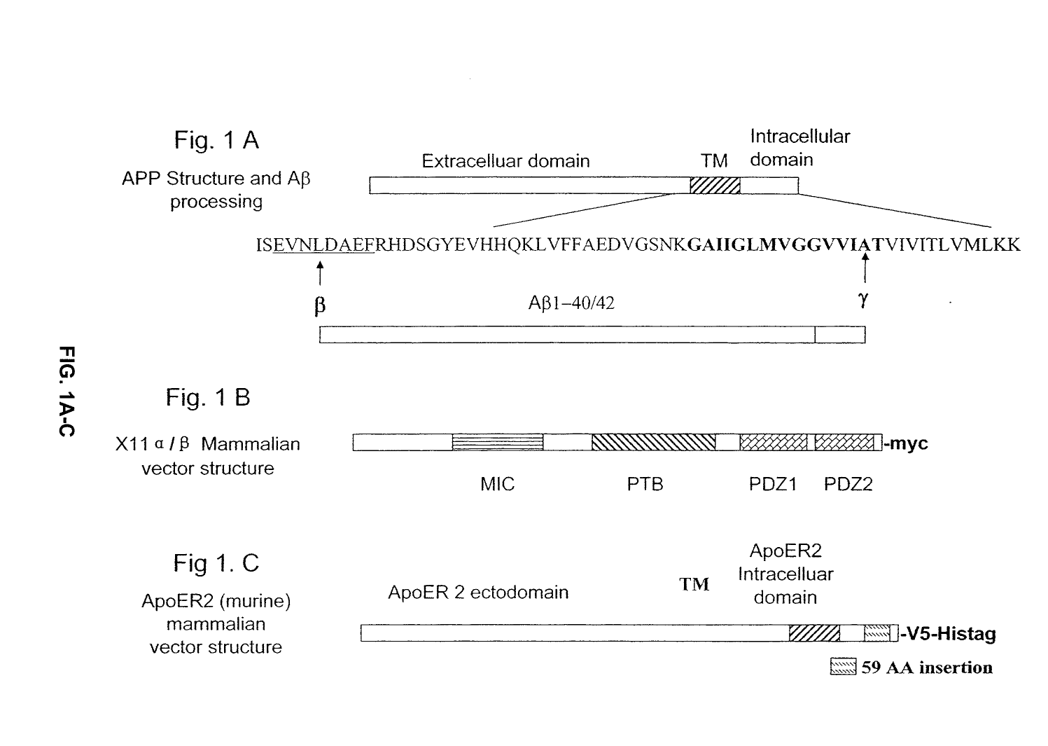

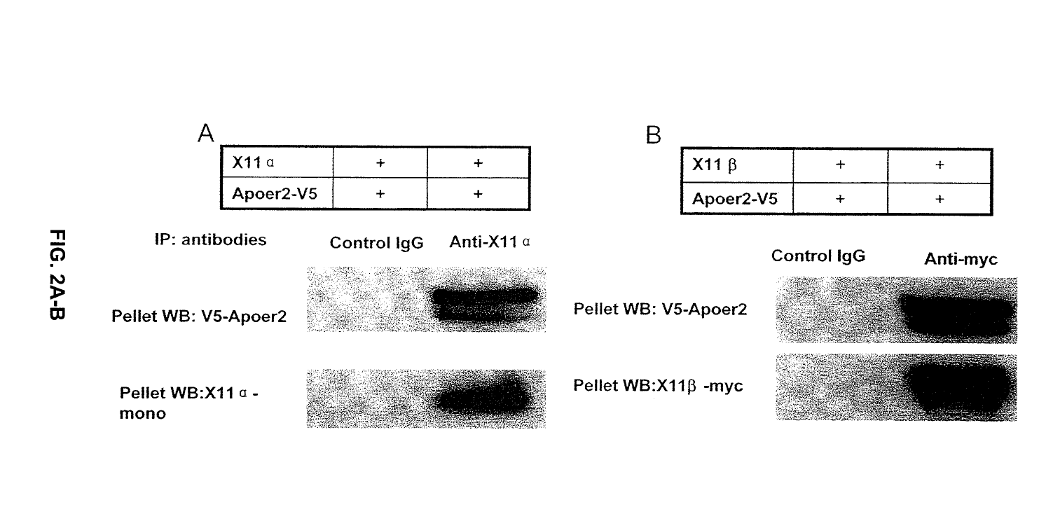

X11 family proteins, X11α/β/γ, (also known as X11, X11-like (X11L) and X11-like 2 (X11L2)) are neuronal adapter proteins. FIG. 1 shows a diagram of the domains in X11 proteins, characterized by phosphotyrosine binding (PTB) domains. While X11γ is ubiquitously distributed in different tissues, X11α and β are expressed only in the brain (Borg et al., 1999; Hase et al., 2002). Each of the family proteins was reported to stabilize intracellular APP and/or suppress Aβ production (King and Turner, 2004). The sequences for X11α and β are provided in SEQ ID NOS:8 and 11, respectively, with SEQ ID NOS:9, 10 and 12 showing various PTB domains.

### IV. Therapeutic Regimens

A. Therapeutic Agents

A variety of therapeutic agents are contemplated for use in accordance with the present invention. Small molecules (biologics or organopharmaceuticals) that mimic the structure/function of ApoE and/or ApoER2 may be designed and tested in accordance with the parameters set forth elsewhere in this document. Other agents, however, can be readily designed and tested based on present information. In particular, these agents are peptides or polypeptides derived from ApoE (including ApoE4), ApoER2, antibodies against these two targets (humanized, single chain, whole or antigen-binding fragments thereof), or nucleic acids encoding these agents. Also contemplated are X11 PTB domains and fragments thereof.

1. Peptides, Polypeptides and Mimetics

Peptides of ApoE maybe designed that are expected to compete with the binding of native ApoE, but that fail to activate the same processes that are invoked by binding of native ApoE to ApoER2. Of particular interest are peptides that span the region of polymorphism in ApoE4, namely, residue 112. Also contemplated are X11 PTB domains and fragments thereof. Peptides will generally be on the order of 6 to 40 amino acids in length, with all intermediate sizes contemplated (6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40). Of these residues at least about 6 will represent consecutive residues from ApoE or X11 PTB domains. However, the number of consecutive residues may again be all intermediate sizes (6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40). The peptide may contain non-ApoE and non-X11 sequences, such as stabilization or targeting domains.

By the same token, peptides from ApoER2 may also act to bind ApoE in solution, thereby reducing its binding to native, surface bound ApoE2. Peptides will generally be on the order of 6 to 40 amino acids in length, with all intermediate sizes contemplated (6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40). Of these residues at least about 6 will represent consecutive residues from ApoER2. However, the number of consecutive residues may again be all intermediate sizes (6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40). The peptide may contain non-ApoER2 sequences, such as stabilization or targeting domains. Alternatively, larger polypeptides including more than 40 residues, up to and including the entire ectodomain of ApoER2 are contemplated.

It may be desired to purify peptides or polypeptides of the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Various techniques suitable for use in protein purification will be well known to those of skill in the art. These include, for example, precipitation with ammonium sulphate, PEG, antibodies and the like or by heat denaturation, followed by centrifugation; chromatography steps such as HPLC, ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

In addition to genetic methods (discussed below), peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart & Young, (1984); Tam et al., (1983); Merrifield, (1986); Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

In addition, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen-membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

2. Antibodies

The term “antibody” is used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibodies and antibody-based constructs and fragments are well known in the art (see, e.g., Harlow et al., 1988; and U.S. Pat. No. 4,196,265 each incorporated herein by reference). The antibody or fragment thereof should exhibit the desired biological activity, i.e., inhibitory of ApoE-ApoER2 interactions. Also contemplated are monoclonal antibodies, polyclonal antibodies, and multispecific antibodies (e.g., bispecific antibodies).

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975), or may be made by recombinant DNA methods (see, e.g., U.S. Pat. No. 4,816,567). The “monoclonal antibodies” may also be isolated from phage antibody libraries using the techniques described in Clackson et al. (1991) and Marks et al. (1991), for example.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

In certain embodiments of the present invention, the nucleic acid of the pharmaceutical compositions and devices set forth herein encodes a single chain antibody. Single-chain antibodies are described in U.S. Pat. Nos. 4,946,778 and 5,888,773, each of which are hereby incorporated by reference.

3. Antisense

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron-exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

4. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

5. RNAi

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25-mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

6. Expression Constructs

Within certain embodiments expression vectors are employed to express a peptide or polypeptide product. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

The term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

7. Viral Delivery

The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Of particular interest in the present invention are neurotrophic viruses such as herpesviruses and lentiviruses. U.S. Pat. Nos. 6,344,445, 5,626,850, 5,223,424, 6,319,703 discuss herpesviruses vectors; U.S. Pat. Nos. 6,924,144, 6,521,457, 6,428,953, 6,277,633, 6,165,782 and 5,994,136 discuss lentivirus vectors.

8. Non-Viral Delivery

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

Recent advances in lipid formulations have improved the efficiency of gene transfer in vivo (Smyth-Templeton et al., 1997, WO 98/07408). A lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

In further embodiments, the liposome is further defined as a nanoparticle. A “nanoparticle” is defined herein to refer to a submicron particle. The submicron particle can be of any size. For example, the nanoparticle may have a diameter of from about 0.1, 1, 10, 100, 300, 500, 700, 1000 nanometers or greater. The nanoparticles that are administered to a subject may be of more than one size.

Any method known to those of ordinary skill in the art can be used to produce nanoparticles. In some embodiments, the nanoparticles are extruded during the production process. Exemplary information pertaining to the production of nanoparticles can be found in U.S. Patent App. Pub. No. 20050143336, U.S. Patent App. Pub. No. 20030223938, U.S. Patent App. Pub. No. 20030147966, and U.S. Provisional Application Ser. No. 60/661,680, each of which is herein specifically incorporated by reference into this section.

B. Combinations Therapies

In another embodiment, the ApoE/ApoER2 inhibitors of the present invention may be used in combination with other agents to improve or enhance the therapeutic effect of either. This process may involve administering both agents to the patient at the same time, either as a single composition or pharmacological formulation that includes both agents, or by administering two distinct compositions or formulations, wherein one composition includes the ApoE/ApoER2 inhibitor and the other includes the second agent(s).

The ApoE/ApoER2 therapy also may precede or follow the other agent treatment by intervals ranging from minutes to weeks. In embodiments where the other agent and ApoE/ApoER2 inhibitor are administered separately, one may prefer that a significant period of time did not expire between the time of each delivery, such that the agent and ApoE inhibitor would still be able to exert an advantageously combined effect. In such instances, it is contemplated that one may administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other embodiments, it may be desirable to alternate the compositions so that the subject is not tolerized.

Various additional combinations may be employed, ApoE/ApoER2 inhibitor therapy is “A” and the secondary agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary.

Various drugs for the treatment of AD are currently available as well as under study and regulatory consideration. The drugs generally fit into the broad categories of cholinesterase inhibitors, muscarinic agonists, anti-oxidants or anti-inflammatories. Galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol and neuromidal are just some of the drugs proposed as therapeutic agents for AD.

### V. Pharmaceutical Formulations and Routes of Administration

Pharmaceutical compositions of the present invention comprise an effective amount of an ApoE/ApoER2 inhibitor and/or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one ApoE/ApoER2 inhibitor or additional active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularlly, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18^{th} Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the compounds of the present invention are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### VI. Screening Methods

The present invention further comprises methods for identifying inhibitors of the ApoE-ApoER2 interaction. These assays may comprise random screening of large libraries of candidate substances; alternatively, the assays may be used to focus on particular classes of compounds selected with an eye towards structural attributes that are believed to make them more likely to interact with and hence modulate the interaction of ApoE-ApoER2.

As used herein the term “candidate substance” refers to any molecule that may potentially inhibit ApoE-ApoER2 binding or subsequent downstream signaling events. The candidate substance may be a peptide, polypeptide, a small molecule, or even a nucleic acid molecule. As discussed above, it may prove to be the case that the most useful pharmacological compounds will be compounds that are structurally related to ApoE and ApoER2, or nucleic acids coding therefor.

Using lead compounds to help develop improved compounds is known as “rational drug design.” The goal of rational drug design is to produce structural analogs of compounds and systematically test these to arrive at suitable compounds with the desired properties. In one approach, one would generate a three-dimensional structure for a target molecule, or a fragment thereof. This could be accomplished by x-ray crystallography, computer modeling or by a combination of both approaches. In another approach, the structural analogs can be modeled after the predicted primary, secondary or tertiary structure of a protein or peptide.

It also is possible to use antibodies to ascertain the structure of a target compound activator or inhibitor, thereby bypassing protein crystallography altogether by generating anti-idiotypic antibodies. As a mirror image of a mirror image, the binding site of anti-idiotype would be expected to be an analog of the original antigen. The anti-idiotype could then be used to identify and isolate compounds from banks of chemicals or biologically-produced peptides. Selected peptides would then serve as the pharmacore. Anti-idiotypes may be generated using the methods described herein for producing antibodies, using an antibody as the antigen. Antibodies and anti-idiotypes to either ApoE or ApoER2 can be used themselves as inhibitors.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators will target the expression of ApoE and ApoER2, and include antisense molecules, ribozymes, and antibodies (including single chain antibodies), each of which would be specific for a target molecule and expressed in a cell that expresses ApoE or ApoER2. A particular antisense molecule that binds to a translational or transcriptional start site, or splice junction of ApoER2 is specifically contemplated.

It will, of course, be understood that all the screening methods of the present invention are useful in themselves notwithstanding the fact that effective candidates may not be found. The invention provides methods for screening for such candidates, not solely methods of finding them.

A. In Vitro Assays

To identify an inhibitor of ApoE-ApoER2, one generally will determine the interaction of ApoE and ApoER2 in the presence and absence of the candidate substance, an inhibitor being defined as any substance that reduces this interaction. For example, a method generally comprises:
(a) providing a candidate modulator;
(b) admixing the candidate modulator with an ApoE and ApoER2 molecule or a cell which expresses ApoER2;
(c) measuring the binding of ApoE to ApoER2; and
(d) comparing the binding measured in step (c) with the binding of ApoE to ApoER2 in the absence of said candidate modulator,
   wherein a reduction in the binding of ApoE to ApoER2 indicates that said candidate modulator is, indeed, an inhibitor of the a ApoE-ApoER2 interaction.

In such assays, the target, be it ApoE or ApoER2, may be either free in solution, fixed to a support, expressed in or in the case of ApoER2 disposed on the surface of a cell. Competitive binding formats can be performed in which one of the agents is labeled, and one may measure the amount of free label versus bound label to determine the effect on binding.

Various readouts for binding can be utilized, for example, alteration in migration patterns on gels, FRET, surface plasmon resonance, or a host of other technologies.

B. In Cyto Assays

The present invention also contemplates the screening of compounds for their ability to inhibit the interaction of ApoE and ApoER2 in intact cells. Various cell lines can be utilized for such screening assays, such as neuronal cells, as well as other specifically engineered for this purpose (e.g., to express or overexpress ApoER2 or to provide a biochemical read-out of ApoER2 ligand binding).

Depending on the assay, culture may be required. The cell may be examined using any of a number of different assays, but in particular, the effect of the candidate substance on Aβ expression in the cell. Alternatively, one may look at ApoER2 expression or translocation within the cell, or ApoER2 mRNA expression, stability or degradation.

C. In Vivo Assays

In accordance with the present invention, there also are provided methods for screening agents or agent combinations for efficacy in treating AD. In an exemplary assay, an ApoE/ApoER2 inhibitor is provided to an experimental animal via an appropriate route. One or more symptoms of AD are then assessed and compared to those seen in a similar animal not receiving the inhibitor, e.g., the same animal prior to receiving the inhibitor. Such symptoms include, but are not limited to decreased locomotor activity, decreased grip strength, inability to perform on water maze or T maze tests, impaired contextual fear conditioned responses. A positive result might be interpreted as the diminution of a symptom, the delay, or prevention in appearance of a previously unseen symptom, or the delay or prevention of progression of an existing symptom.

The method may also comprise screening an ApoE-ApoER2 inhibitor in combination with another agent. Thus, depending on whether one was more interested in examining the inhibitor, the other agent or the combination, the appropriate control would be an animal untreated with the inhibitor, the other agent, or both, respectively. The assay may also comprise various other parameters, including timing of administration, varying the dose, assessing toxicity.

Mouse models with clinical features suggestive of AD have been generated. The amyloid β (A4) precursor protein (APP) targeted mutation mice were generated by Dr. David Borchelt and can be purchased from The Jackson Laboratory (Bar Harbor, Me.). This mouse model develops decreased forelimb grip strength and locomotor activity. In addition, reactive astrocytosis can be demonstrated by histopathology by 14 weeks of age. The double transgenic APP (chimeric-mouse/human)-presenilin 1 (human), also generated by Dr. David Borchelt, can also be obtained from the Jackson Laboratory. The latter mice start accumulating amyloid deposits in the brain by nine months of age, similar to those found in human AD brains. These deposits increase dramatically by age 12 months. AD mouse models develop behavioral alterations that can be assessed using various tests, including the water maze, T maze, or contextual fear conditioning tests. Thus, a drug proposed to ameliorate AD in humans can be assessed and validated on the AD animal models. Other AD mice with various levels of expression of APPs have been generated, including animals that develop signs of disease or synaptic toxicity prior to plaque formation (Mucke et al., 2000). Models with the various mutations leading to AD-like pathology are reviewed in Price and Sisodia (1998). This model will find use in screening of compounds according to the present invention for activity against AD and symptoms thereof.

### VII. Identifying Subjects having AD

In various aspects of the invention, it will be desirable to identify subjects that have AD. The general approaches for diagnosis is set out below. It also may be desirable to identify those individuals having increased risk for AD. At present, there are no truly prognostic tests. However, any of the following diagnostic procedures may be applied to individuals with few or no overt symptoms of AD and, in this way, provide early treatment that may prevent related neuropathologic damage and/or progression of the disease to a more clinically significant stage.

In various aspects of the invention, it will be desirable to identify subjects that have AD. The general approaches for diagnosis of these diseases are set out below. It also may be desirable to identify those individuals having increased risk for AD. At present, there are no truly prognostic tests. However, any of the following diagnostic procedures may be applied to individuals with few or no overt symptoms of AD and, in this way, provide early treatment that may prevent related neuropathologic damage and/or progression of the disease to a more clinically significant stage.

The diagnosis of both early (mild) cognitive impairment and AD are based primarily on clinical judgment. However, a variety of neuropsychological tests aid the clinician in reaching a diagnosis. Early detection of only memory deficits may be helpful in suggesting early signs of AD, since other dementias may present with memory deficits and other signs. Cognitive performance tests that assess early global cognitive dysfunction are useful, as well as measures of working memory, episodic memory, semantic memory, perceptual speed and visuospatial ability. These tests can be administered clinically, alone or in combination. Examples of cognitive tests according to cognitive domain are shown as examples, and include “Digits Backward” and “Symbol Digit” (Attention), “Word List Recall” and “Word List Recognition” (Memory), “Boston Naming” and “Category Fluency” (Language), “MMSE 1-10” (Orientation), and “Line Orientation” (Visuospatial). Thus, neuropsychological tests and education-adjusted ratings are assessed in combination with data on effort, education, occupation, and motor and sensory deficits. Since there are no consensus criteria to clinically diagnose mild cognitive impairment, various combinations of the above plus the clinical examination by an experienced neuropsychologist or neurologist are key to proper diagnosis. As the disease becomes more manifest (i.e., becomes a dementia rather than mild cognitive impairment), the clinician may use the criteria for dementia and AD set out by the joint working group of the National Institute of Neurologic and Communicative Disorders and Stroke/AD and Related Disorders Association (NINCDS/ADRDA). On occasion, a clinician may request a head computed tomography (CT) or a head magnetic resonance imaging (MRI) to assess degree of lobar atrophy, although this is not a requirement for the clinical diagnosis.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

cDNA Vectors. Swedish mutant of APP cDNAs were previously described (Lin et al., 2000). The vectors of human X11α, X11β, and murine ApoER2 were provided from Dr. Chistopher C. J. Miller (Kings College London, UK), Dr. Ben Margolis (University of Michigan), and Dr. Johannes Nimpf (University of Vienna, Austria). PCR was used to transfer the murine ApoER2 gene into pcDNA6.1 with V5-His-Tag at the C-terminal for detection.

Antibodies and Purified Apolipoproein. Mouse monoclonal anti-APP MAB5228 and mouse IgG were bought from CHEMICON (Temecula, Calif.). Mouse monoclonal antibody for X11α was purchased from BD Biosciences. Monoclonal V5 antibody was purchased from Invitrogen. Purified ApoE and ApoE4 were purchased from Biodesign Company.

Co-immunoprecipitation and Western Blot Assays. Human embryonic kidney 293 cells (HEK293) were transfected as described previously (He et al., 2005) with the indicated combinations of plasmids for ApoER2/X11α, ApoER2/X11β, or ApoER2/X11α/APP using Lipofectamine-2000 (Invitrogen). 36 h after transfection, cells were harvested and lysed on ice in lysis buffer (PBS, pH 7.4 with 1% NP-40 and 1% Saponin with protease inhibitors cocktail (Roche)). Cell lysates were cleared by centrifugation and immunoprecipitated with indicated antibody for 4 h at 4° C. Immune complexes were isolated using protein G-agarose beads (Sigma) and subjected to SDS-PAGE and Western blotting using specific antibodies.

Quantification of β-Amyloid40, Neuro-2a cell stably overexpressing APPsw695 was used for detecting of β-Amyloid40. N2a-APPsw cells were grown in Dulbecco's Modified Eagle's medium (DMED) containing 10% fetal bovine serum, 100 U/mL penicillin, and 100 μg/mL streptomycin (GibcoBUR) with 80 μg/mL G418. Cells were cultured in 24-well plates one day before treatment. Transient transfections were done using FeGENE6 (Roche), fresh Optimum medium (Invitrogen) were supplied 5 h after transfection. Same concentration purified Apolipoprotein was added into the fresh medium. Conditioned medium was collected 24 h later. Aβ40 was quantified with sandwich ELISA by using Aβ40 ELISA Kit (Biosource). At least three times assays were done for each condition.

### Example 2

### Results

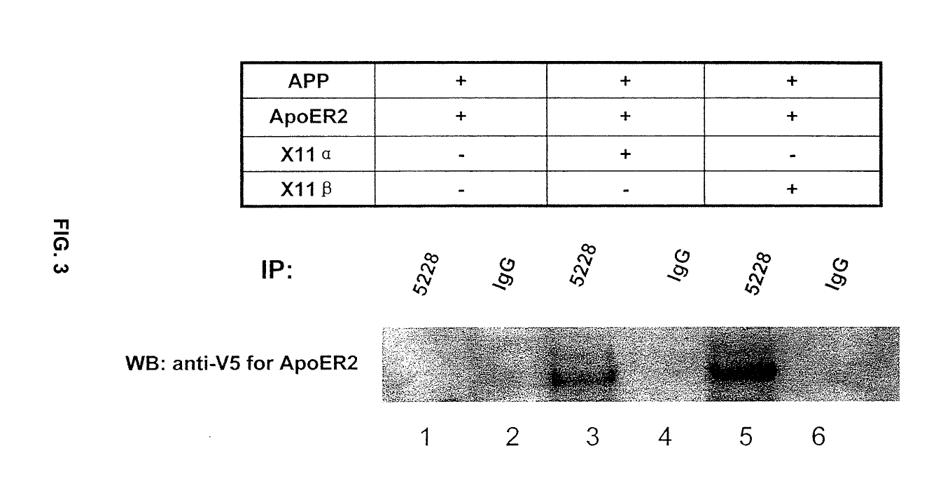

ApoER2 binding with X11α/β. The inventors have demonstrated that ApoER2 binds X11, either α or β form. In these experiments, lysates of 293 cells transiently expressing ApoER2 was subjected to immuno-precipitation with either anti-X11α/anti-myc for X11β. FIGS. 2A-B shows that SDS-PAGE and Western blots for ApoER2 using an anti-V5 antibody (V5 is part of the ApoER2 fusion construct) revealed a doublet band of ApoER2. These results demonstrate that X11α or X11β binds to ApoER2. Since X11 protein is located in the cells, the area of ApoER2 interacts with X11 is likely the intracellular domain. It may be noted here that APP has been demonstrated to bind X11 through its intracellular domain.

Complex formation among APP, ApoER2, X11α/β. The inventors found that ApoER2 forms complex with APP in the presence of either X11α or X11β. In these experiments, human embryonic kidney 293 cells were transfected to express APP with ApoER2 or APP, ApoER2 with X11α/β. Cell lysates from each group was precipitated with anti-APP antibody 5228. The pellets were separated by SDS-PAGE and detected for ApoER2 by Western blot using an anti-V5 antibody. The Western blot patterns in FIG. 3 show that co-expression of APP, ApoER2 and either X11α or X11β resulted in the co-immunoprecipitation of ApoER2 with APP. Controls, in which anti-APP antibody 5228 was substituted with a non-specific IgG fraction, did not reveal ApoER2 bands in Western blots, indicating that the co-immunoprecipitation of ApoER2 is specific association with APP. Cells expressing only APP and ApoER2, but were without either X11α or X11β, did not result in co-immunoprecipitation of ApoER2. These observations indicated that X11α or X11β mediates the complex formation of APP and ApoER2.

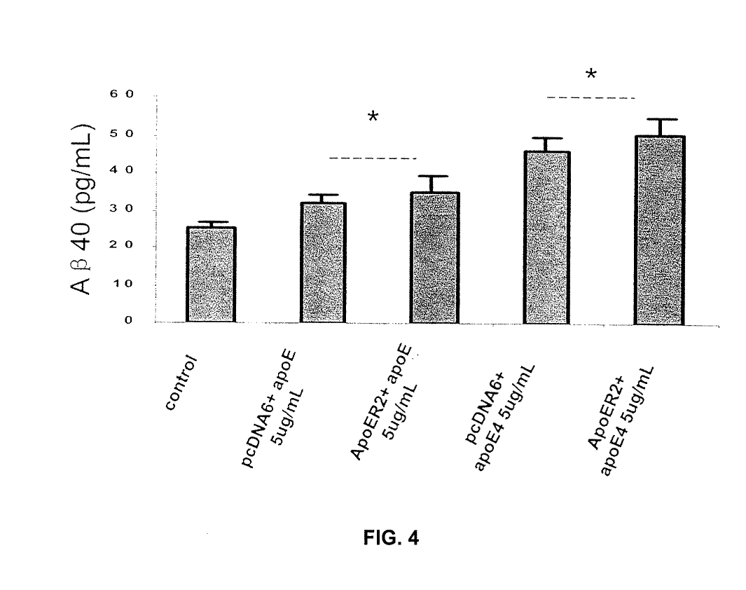

ApoE increases Aβ secretion mediated by ApoER2. The inventors have found that the addition of apolipoprotein E or apolipoprotein E4 to cultured neuronal cell line stimulates the secretion of Aβ40 into culture medium. In these studies, mouse neuroblastoma cells N2a were stably transfected with APPsw. ApoE or ApoE4 was added into OPTIM medium at a concentration of 5 μg/mL and the level of Aβ40 in the media measured. FIG. 4 shows that the addition of ApoE or ApoE4 caused the increase of Aβ40 at a greater extent in the cells transfected with ApoER2 than those transfect with blank vectors. The differences between the Aβ40 levels in cells with and without ApoER2 transfection were statistically significant (p<0.05). These results demonstrate that ApoER2 mediates the increase of Aβ40 in the presence of Apo E, especially Apo E4. FIG. 4 also shows that Aβ40 levels increased over that in the control cells by the addition of ApoE or ApoE4 to the blank vector transfected cells. The endogenous ApoER2 may also mediate this increase.

### Example 3

### Materials & Methods

cDNA vectors. Vectors for human β-secretase and Swedish mutant of APP were previously described (Lin et al., 2000). Vectors for human X11α, X11β, human LRP, and murine ApoER2 were kindly provided by Dr. Chistopher C J Miller (Kings College London, UK), Dr. Ben Margolis (University of Michigan), Dr. Guojun Bu (Washington University), and Dr. Johannes Nimpf (University of Vienna, Austria) respectively. PCR was used for transferring murine ApoER2 gene into pcDNA6 with V5-His-Tag at the C-terminus and for creating mutants of murine ApoER2 in pcDNA6. PTB domain of X11α (residue 457-751) were amplified by PCR and inserted into pcDNA6. GST constructs of GST-PTB (457-751), GST-PDZ (650-837) of X11α and GST constructs of ApoER2 cytosolic domain GST-ApoC1 (881-996), GST-ApoC2 (881-925), GST-ApoC3 (926-996) and GST-APPc (650-695) were amplified by PCR and inserted into plasmid pGEX6.1 (Pharmacia). All constructs were verified by sequencing. GST fusion proteins were produced in Escherichia coli BL21 strain and purified as described previously (He et al., 2003).

Antibodies. Goat polyclonal antibodies against recombinant pro-β-secretase were affinity-purified using Affigel (Bio-Rad) immobilized recombinant β-secretase. Rabbit polyclonal APP antibody AB5228 and mouse IgG were obtained from Chemicon. Mouse monoclonal antibody for X11α was purchased from BD Biosciences. Mouse monoclonal antibody for LRP was purchased from Calbiochem. Monoclonal V5 antibody, Myc antibody and polyclonal antibody for ApoER2 were purchased from Invitrogen. Rabbit polyclonal anti-β-actin was purchased from Novus Biologicals. Monoclonal apoE and apoE4 antibodies were purchased from MBL.

Lipoproteins. ApoE, and ApoE4 were from Biodesign; ApoE, ApoE2, ApoE3, and ApoE4 were from Alpha Diagnostic. Human plasma lipoprotein fractions were prepared as follows: Out dated plasma was obtained from the Oklahoma Blood Bank and was supplemented with antibiotics, preservatives and phenylmethylsulfonyl fluoride to prevent oxidative and proteolytic damage (Manchekar et al., 2004). Very low density lipoprotein (VLDL, d<1.006 g/ml), low density lipoprotein (LDL, d 1.006-1.063) and high density lipoprotein (HDL, d 1.063-1.21 g/mL) were prepared by sequential ultracentrifugation and dialyzed against PBS. The contents of ApoE4 and total ApoE were analyzed for individual VLDL samples using Western blots with monoclonal antibodies. Quantity of bands was determined by optical scanning using STORM Scanner (Amersham Biosciences) and ImageQuant (Molecular Dynamics).

Immunoprecipitation and Western blots. HEK293 cells were transfected as described previously (He et al., 2005) with the following combinations of plasmids: ApoER2/X11α, ApoER2/X11β, or ApoER2/X11α/APPsw695. Cells were harvested 36 h after transfection and lysed on ice in lysates buffer (PBS, pH 7.4 with 1% NP-40 and 1% Saponin with protease inhibitors cocktail (Roche)). Cell lysates were cleared by centrifugation and incubated with indicated antibody for 4 h at 4° C. Immune complexes were isolated using protein G-agarose beads (Sigma) and subjected to SDS-PAGE and Western blotting using specific antibodies as indicated.

Pull-down assays. For GST pull down, GST fusion proteins were retrieved as previously described (He et al., 2003). The amount of protein recovered with glutathione beads was determined using Bio-Rad Protein Assay and diluted to yield a concentration of 1 mg protein/mL of beads. Cell lysates from HEK293 cells transfected with X11α or X11β plasmids were cleared by centrifugation and aliquots of the supernatant were incubated with the same amount of GST fusion protein bound to glutathione beads in PBS buffer for 4 h at 4° C. For NI-NTA (nickel-nitrilotriacetic acid) pull-down experiments, lysates from HEK 293 cells overexpressed X11α, X11α/ApoER2, X11β, or X11β/ApoER2 were individually incubated with same amount NI-NTA beads (Qiagen) in PBS for 4 h at 4° C. The bound proteins on both types of pull-down beads were recovered with SDS sample buffer at boiling temperature and subjected to Western blot.

Cellular Aβ40 production. Neuroblastoma N2a-APPsw cells (from Dr. Riqiang Yan, Lerner Research Institute, Cleveland, Ohio) were cultured in 24-well plates in Dulbecco's Modified Eagle's medium (DMED) containing 10% fetal bovine serum, 100 U/mL penicillin, and 100 μg/mL streptomycin (GibcoBUR) with 80 μg/mL G418 one day before use. Transient transfections were done using FeGENE6 (Roche), fresh Optimum medium (Invitrogen) were supplied 5 h after transfection. Apolipoproteins were added in the fresh medium at 10 μg/mL. Conditioned medium was collected 24 h later. Aβ40 was determined in tripcates using Aβ40 ELISA Kit (Biosource). For studying the effect of PTB domain on Aβ production, N2a-APPsw cells were transfected with (0.4 μg/per well for 24 well plate) of PTB-containing vector before subjected to the above procedure.

Internalization of cell surface proteins. N2a-APPsw cells were transiently tranfected with ApoER2 and β-secretase with Lipfectamine 2000. Twenty four h after transfection, 10 μg/mL ApoE, or ApoE4 was add into medium and incubated for 2 h. Cells were then placed on ice, rinsed in cold PBS and incubated in PBS containing 1.5 mg/mL sulfo-NHS-LC-biotin (Pierce) for 20 min at 4° C. Cells were rinsed twice in PBS and lysed in 800 μl PBS containing 0.1% SDS, 1% NP40 and a complete protease inhibitor cocktail. Total protein concentration was determined by immunoblotting from an aliquot of 50 μl cell lysate. Biotinylated proteins were recovered with NeutrAvidin agarose (50 μl; Pierce), rinsed three times, eluted in 20 μl SDS-sample buffer at boiling temperature and used for Western-blot. Quantity of bands was determined by optical scanning instruments specified above. For the determination of internalized proteins, cells were prepared as above, rinsed in PBS and then incubated with cold 1.5 mg/mL cleavable biotin reagent in PBS (EZ-Link Sulfo-NHS-SS biotin, Pierce) for 20 min at 4° C. Cells were then rinsed quickly with medium at room temperature, incubated for 15 min at 37° C. in control medium or in medium containing 10 μg/mL ApoE, or ApoE4, placed on ice and rinsed in cold stripping buffer (50 mM glutathione, 75 mM NaCl, 75 mM NaOH, 10% FBS, pH 8.5-9.0). Cells were then lysed in 800 μl PBS containing 0.1% SDS, 1% NP40 and a complete protease inhibitor cocktail. After centrifugation, biotinylated proteins were retrieved by incubation with NeutrAvidin agarose (50 μl; Pierce). Isolated proteins were rinsed in buffer three times and boiled in 20 μl sample buffer for Western blots.

Immuno-fluorescence studies of cellular proteins. HeLa cells were seeded onto 6-well plates with glasses coverslips, and expressed the ApoER2/X11α, ApoER2/X11β, or ApoER2/APP constructs for 36 h after transfection. Cells were gently fixed in 4% paraformaldehyde, in phosphate-buffered saline, pH 7.4, at room temperature for 15 min. Coverslips were then washed twice for 10 min each in PBS and incubated for 1 h at room temperature with the indicated combinations of primary antibodies diluted in 0.1% BSA, 0.1% saponin, 0.02% sodium azide in PBS (immunofluorescence buffer). At the end of this period, coverslips were washed twice with PBS followed by incubation for 30 min with the indicated combinations of secondary antibodies diluted in immunofluoresence buffer. Coverslips were again washed twice with PBS and mounted on slides using Vector-shield (Vector Laboratories, Inc., Burlingame, Calif.). APP was immunolabeled with Rabbit polyclonal antibody 5352 (CHEMICON) and followed by Cy3 conjugated sheep anti-rabbit secondary antibody (Sigma). X11α and X11β were blotted by monoclonal antibodies again them (BD Biosciences) and then recognized by Alexa-488 conjugated donkey anti-mouse secondary antibody (Invitrogen). ApoER2 was labeled either by Rabbit polyclonal antibody against His-tag (Colocalize with X11) or monoclonal antibody against V5 (Colocalize with APP), and then was labeled either Cy3 or Alexa-488 conjugated secondary antibodies. Images were obtained in an inverted confocal laser scanning microscope (LSM 510, Carl Zeiss Inc.).

SiRNA interference of ApoER2. Double-stranded siRNA specific for the mouse ApoER2 (ON-TARGETplus SMARTpool, Catalog No. L-046407-00) were chemically synthesized by Dharmacon. N2a-APPsw cells grown in 24-well plates for 24 h were transfected with either ApoER2 siRNA or control siRNA (No-Target siRNA, Dharmacon) with Oligofectamine (Invitrogen). The medium, Opti-MEM, was replaced 4 h later and ApoE or ApoE4 and cultured for 24 h. Aβ40 in the medium samples were assayed as described above.

Studies on Reelin and Reelin effect. N2a-APPsw cells grown in a T25 flask were transfected with 5 μg of expression vector pCr1 containing the entire mouse Reelin gene using Lipofectamine-2000. Control cells were transfected with blank vector. Cells were conditioned in OPTI-MEM for 36 h and the medium was collected and concentrated. Western blot was carried out on concentrated medium and cell lysate using anti-Reelin antibody MAB5364 (CHEMICON). To determine the effect of Reelin antibody on Aβ production, N2a-APPsw cells were cultured for 24 h with 10 μg/mL ApoE and either with or without antibody MAB5364. Aβ40 in the medium was determined by ELISA.

### Example 4

### Results

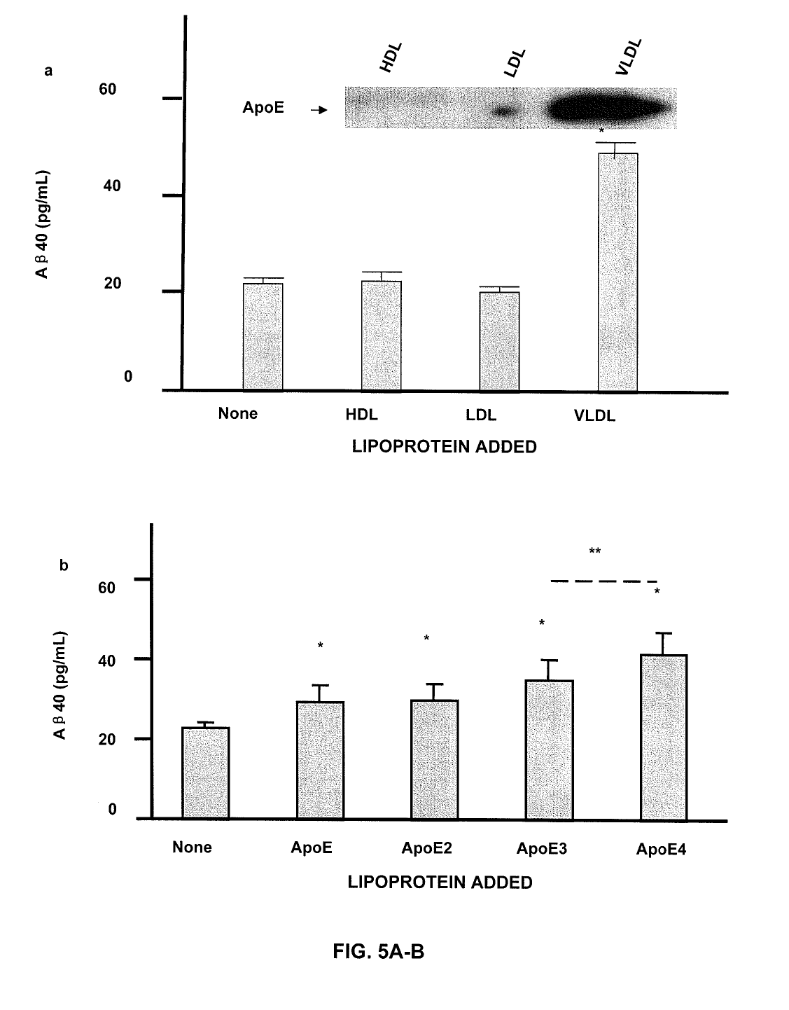

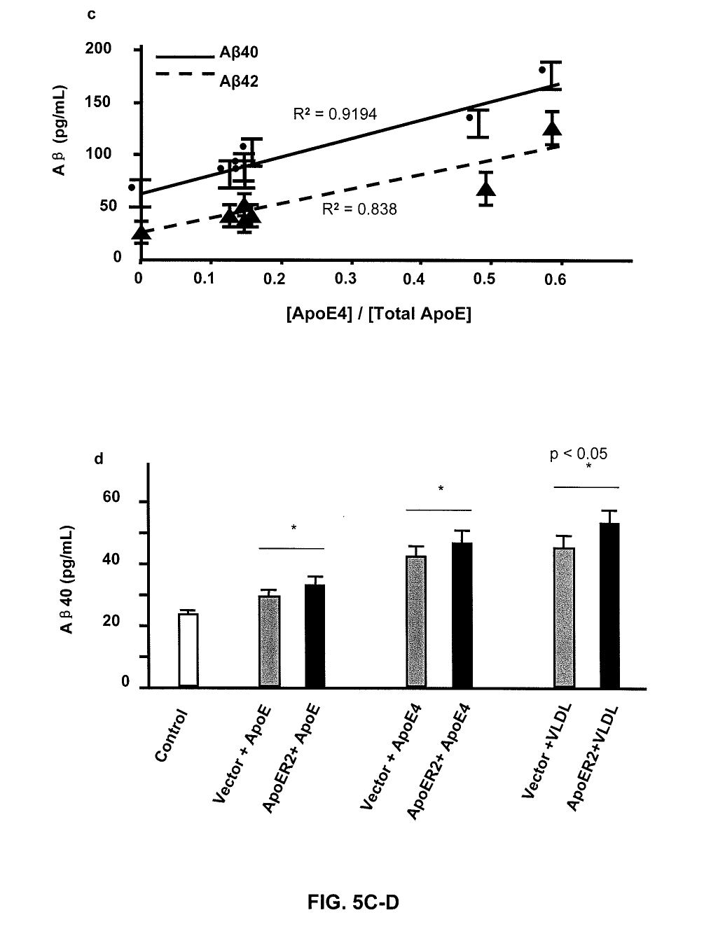

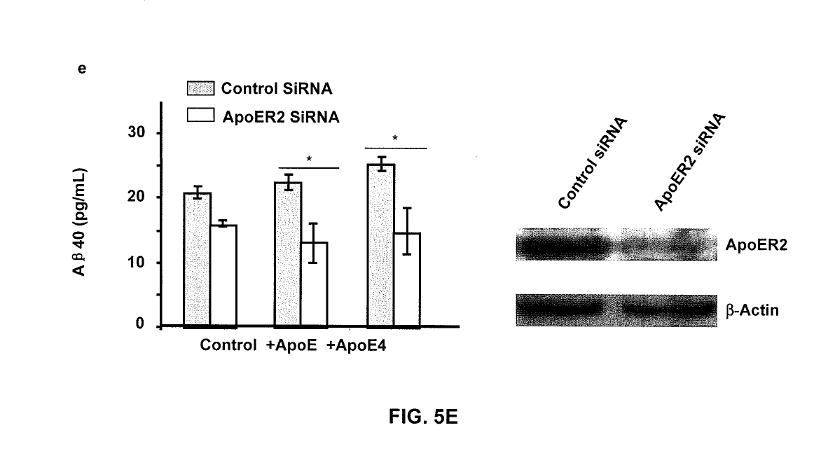

ApoE increases Aβ in N2a cells. The inventor observed that Aβ production in Neuro-2a cells stably transfected with human APPswedish (N2a-APPsw) increased up to two folds in the presence of purified human VLDL (p<0.01) while LDL and HDL had no effect (FIG. 5A). Since VLDL is the main lipoprotein fraction contains ApoE (FIG. 1A, inset), the inventor tested the effect of each of the isomorphic ApoE forms and found that the addition of recombinant ApoE2, ApoE3 and ApoE4 to the cell culture at 10 μg/ml increased Aβ production (FIG. 5B) with the highest response by ApoE4. Since these ApoE samples contained no lipids, the differential response may be influenced by their non-native physical properties. The inventor therefore investigated if ApoE4 from native human VLDL stimulates more Aβ production than either ApoE2 or ApoE3. VLDL was prepared from individual human plasma and the content of total ApoE and ApoE4 was determined. VLDL samples containing different levels of ApoE were chosen for measuring the production of Aβ₄₀ and Aβ₄₂ in N2a-APPsw cells. The inventor observed a linear correlation of ApoE4 content and the production of both Aβ species (FIG. 5C) with similar correlation coefficients. These results indicate that ApoE4 in native lipoprotein particles also stimulates higher Aβ production in N2a cells than those for ApoE2 and ApoE3.

ApoER2 mediates Aβ increase by ApoE. Since ApoER2 is a brain-specific receptor for ApoE, the inventors asked if the ApoE effect on Aβ is mediated by ApoER2. In the first set of experiments, the influence of Apo E and VLDL on Aβ production was compared for N2a-APPsw cells with or without transfection of ApoER2. Cells transfected with ApoER2 produced more Aβ in response to ApoE, recombinant ApoE4 or purified VLDL (FIG. 5D). These results suggest that ApoER2 mediated the increase of Aβ production in response to ApoE. ApoE also caused Aβ to increase in cells transfected by blank vector (FIG. 5D) which provided an opportunity to study if the increase was mediated by endogenous ApoER2. In the second series of experiments, the inventor employed siRNA to knock-down ApoER2 and examined Aβ production in response to the various ApoE species. FIG. 5E shows that only residual expression of endogenous ApoER2 remained in knock-down cells, which lost about one-fourth of their Aβ production and, which did not increase upon the addition of ApoE or ApoE4 to the media (FIG. 5D). These observations indicate that ApoER2 mediates ApoE stimulated Aβ production in N2a-APPswedish cells.

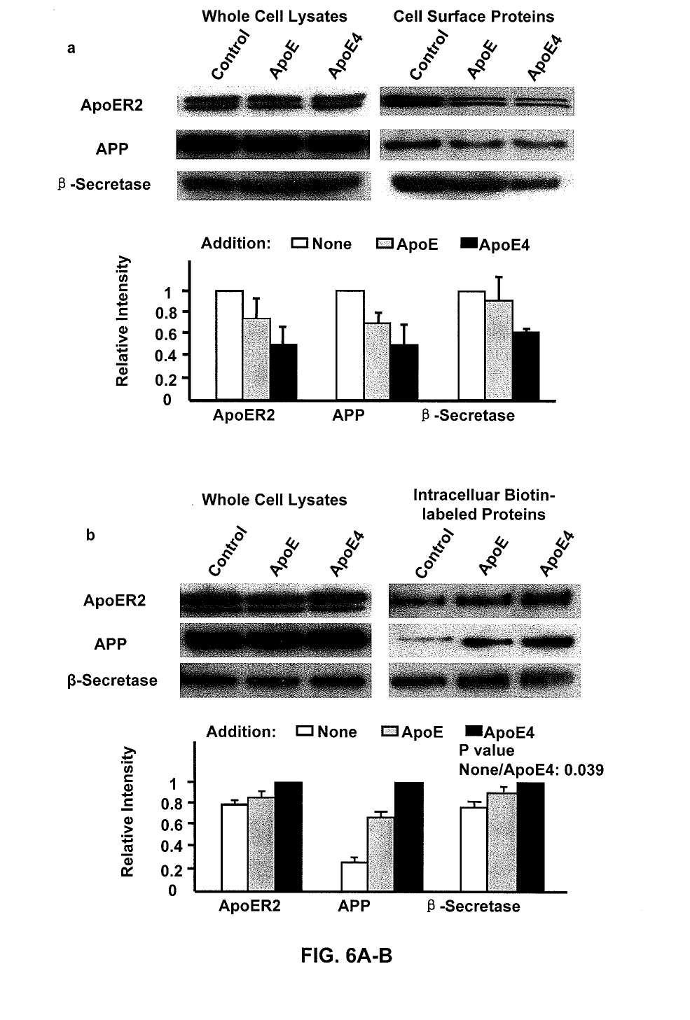

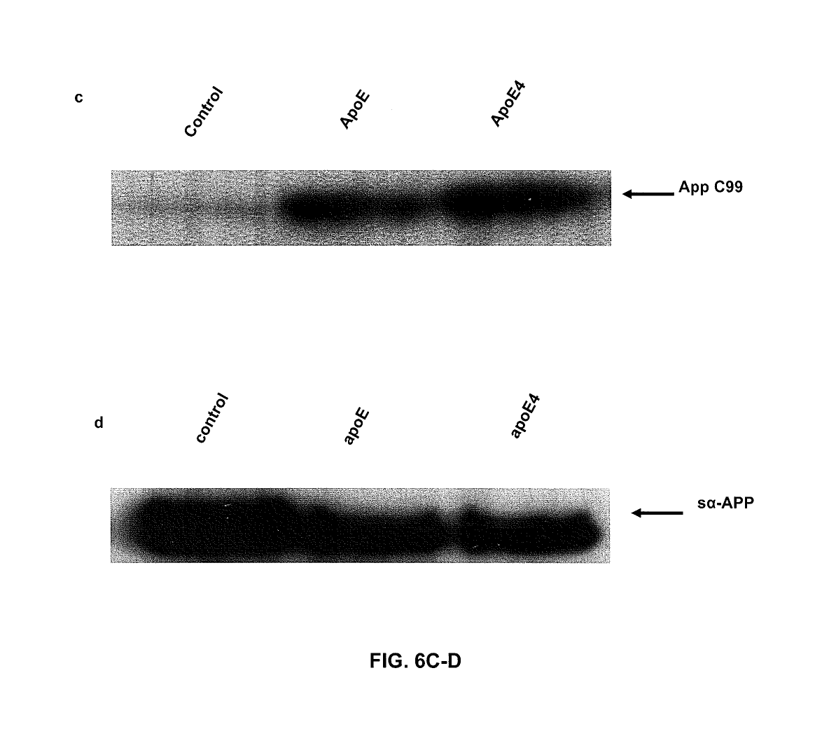

ApoE triggers the endocytosis of ApoER2, APP and β-secretase. A possible explanation for ApoE stimulation of Aβ production is that ApoE triggers the internalization of not only ApoER2, but also APP and β-secretase from cell surface to endosomes. The inventor examined if the addition of ApoE causes the reduction of these proteins from the cell surface. N2a-APPswedish cells were treated with ApoE or ApoE4 for 2 h and then biotinylated to label the cell surface proteins. Biotinylated ApoER2, APP and β-secretase were recovered and revealed in Western blot. As compared to controls, the remaining cell-surface ApoER2, APP and β-secretase decreased by about 25%, 25% and 7% respectively when ApoE was added and 50%, 50% and 35% respectively when ApoE4 was present (FIG. 6A). The total amounts of the three proteins in the cell lysates remained about the same. The inventor then determined if ApoEs increased these three proteins inside the cells. Cell-surface proteins were pulse-labeled by biotinylation and permitted to internalize for 15 min. Biotinyl groups remained at cell surface were stripped and the biotinylated ApoER2, APP and β-secretase inside the cells were retrieved and visualized in Western blot. The inventor observed small increases of ApoER2 and β-secretase in response to ApoE and ApoE4 (FIG. 6B) while the increase of APP was much greater, about 2.5- and 4-fold in response to ApoE and ApoE4, respectively (FIG. 6B). Together, the depletion of cell-surface ApoER2, APP and β-secretase and the increase of the intracellular pool of these proteins in response to the addition of ApoE are consistent with the mechanism that the binding of ApoE to ApoER2 triggered the endocytosis of all three proteins. To further substantiate the endocytosed APP was subjected to proteolysis by β-secretase, the inventor also determined in the above experiments the APP C-terminal fragment resulting from β-secretase cut (C99). The inventor observed that C99 was significantly increased in the presence of ApoE and ApoE4 over the control (FIG. 6C). Again, ApoE4 generated more C99 than did ApoE. An increased endocytosis and decreased cell-surface APP stimulated by ApoE should be accompanied by a decrease of APP processing by ∝-secretase. Western blots of the APP ectodomain generated by ∝-secretase, s∝-APP, confirmed a reduction of s∝-APP by ApoE and ApoE4 (FIG. 6D).

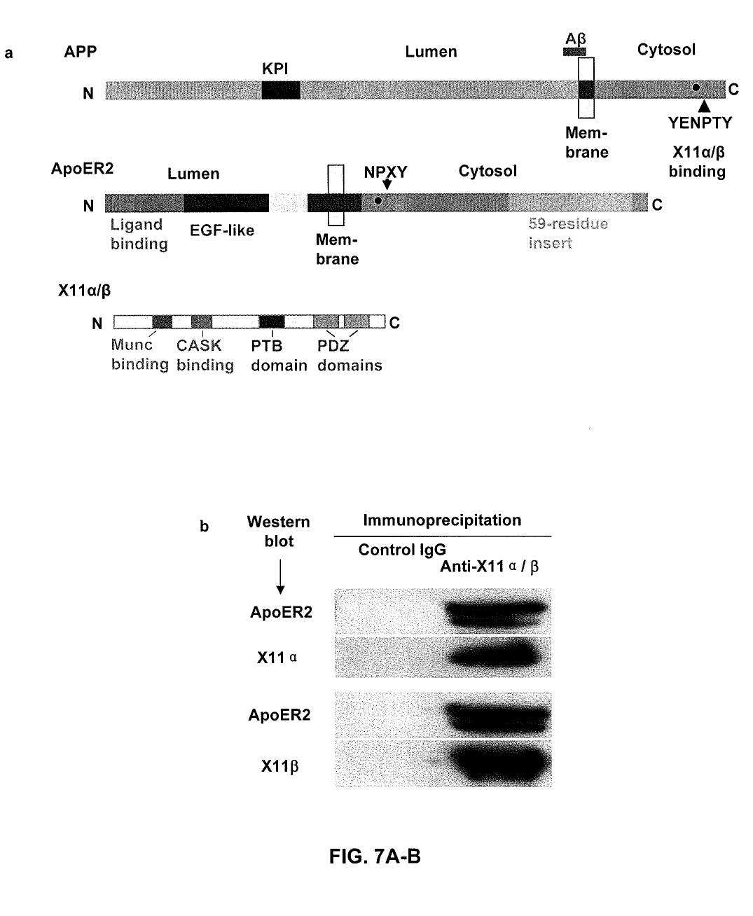

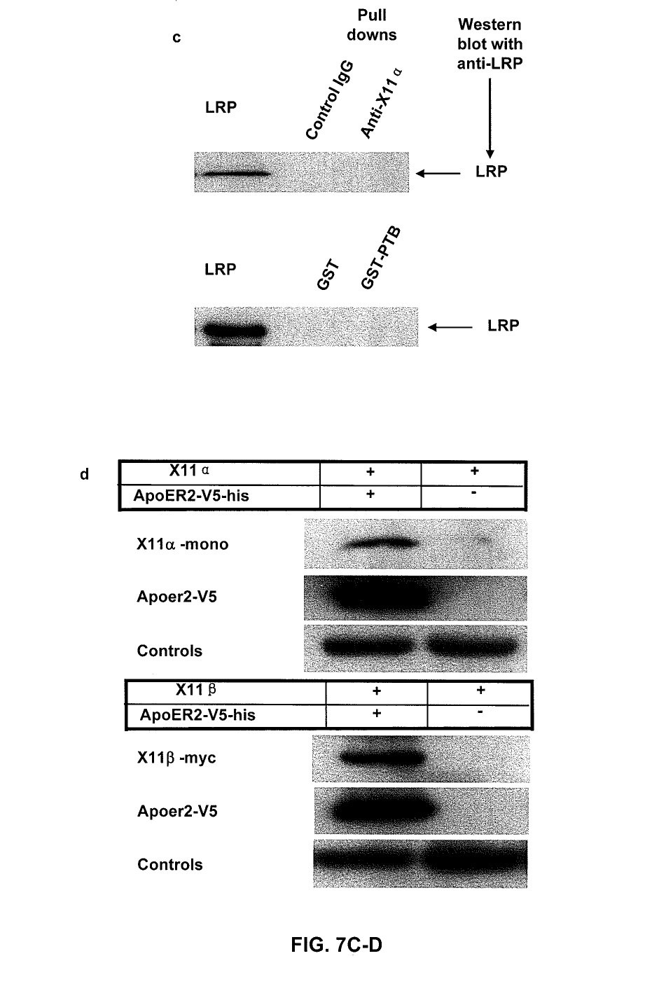

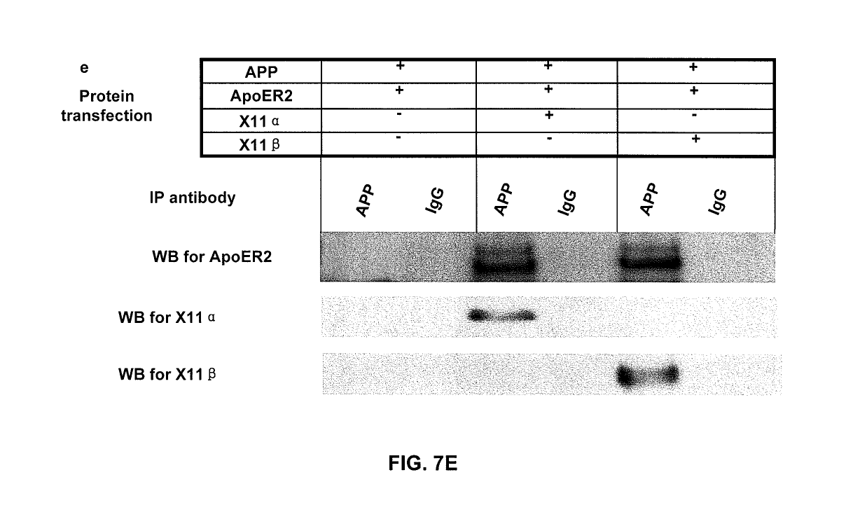

Role of X11α/β in the ApoE and Aβ relationship. A hypothesis that may explain the ApoE triggered endocytosis of ApoER2 and APP is that their cytosolic domains may interact with a same adaptor protein that regulates cellular transport. The cytosolic domain of APP contains a YENPTY motif known to bind brain specific adaptor proteins X11α or X11β (FIG. 7A). The inventor seek to determine if X11α/β also interacts with ApoER2. When ApoER2 was immuno-precipitated from lysates of HEK293 cells transiently expressing ApoER2 and either X11α or X11β, the adaptor proteins were seen in the Western blots of the precipitate (FIG. 7B). Substitution of ApoER2 with LRP did not produce co-immuno-precipitation with X11α or the ApoER2-binding PTB domain of X11α (see below) (FIG. 7C), indicating that X11 binding to ApoER2 is specific rather than a general phenomenon with LDL receptor family. X11α and X11β gave essentially identical results. X11γ was not tested as it is not expressed in the brain. In reverse pull down, ApoER2 containing a His6 tag in the lysate collected with Ni-affinity gel also recovered X11α/β (FIG. 7D). These results suggest that X11α/β binds ApoER2 with high affinity. Since X11α/β binds both ApoER2 and APP, the inventor tested if ApoER2 and APP form a complex in non-neuronal HEK293 cells devoid of these endogenous proteins of interest. When the lysates of cells expressing ApoER2, APP and X11α/β were immunoprecipitated with anti-APP antibody 5228, both APP and X11 were found in the Western blot of the precipitates (FIG. 7E). Again, both X11α and X11β supported the coprecipitation. Cells transfected with ApoER2 and APP but without X11 had virtually no coprecipitation of ApoER2 and APP (FIG. 7E). These results indicate that X11α/β is required for the complex formation involving ApoER2, APP and X11α/β.

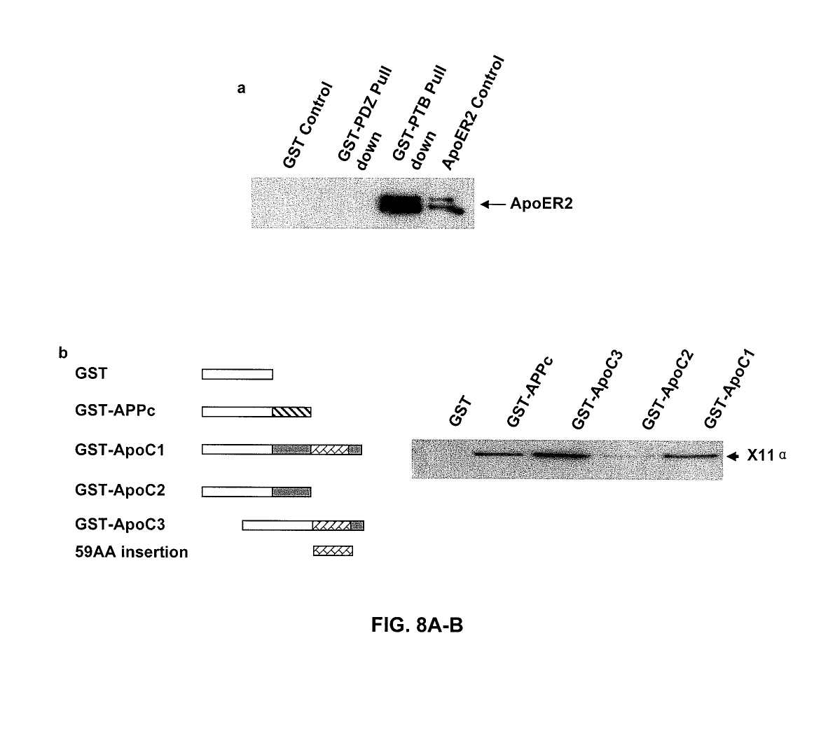

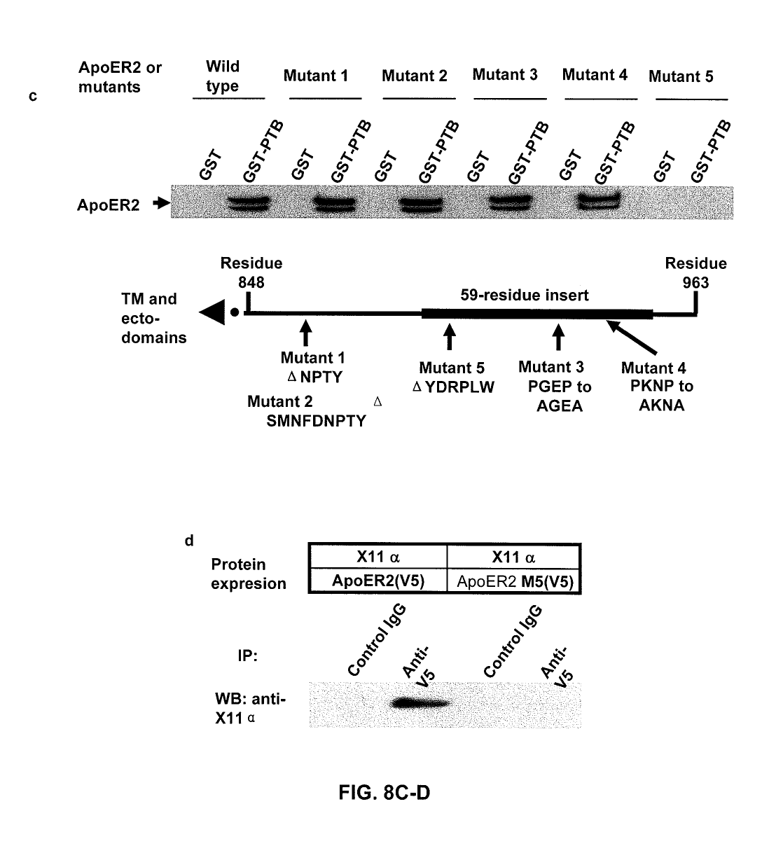

Domain interaction between ApoER2 and X11α/β. PTB domain of X11α but not the PDZ domain was shown to pull down ApoER2 from the cell lysate (FIG. 8A), indicating that it is responsible for ApoER2 binding. To identify the binding motif in ApoER2, various GST-fusions from different regions of ApoER2 cytosolic domain were tested for X11α/β pull down. Constructs contained the 59-residue insertion were able to bind X11 (FIG. 8B) indicating that this region contains an X11 binding motif. Various deletions or mutations were made for potential motif sites in the cytosolic domain of ApoER2. Deletions of a NPXY motif (mutants 1 & 2, FIG. 8C) or mutations of two PXXP motifs (mutants 3 & 4, FIG. 8C) did not affect the binding of X11-PTB domain to ApoER2. The deletion of residues YDRPLW (residues 899-904) within the 59-residue insertion abolished X11-PTB pull-down (mutant 5, FIG. 8C). This same deletion also abolished the pull down of a separately expressed PTB domain of X11α (FIG. 8D). These results indicate that the motif YDRPLW mediates the binding of ApoER2 to the PTB domain of X11α/β.

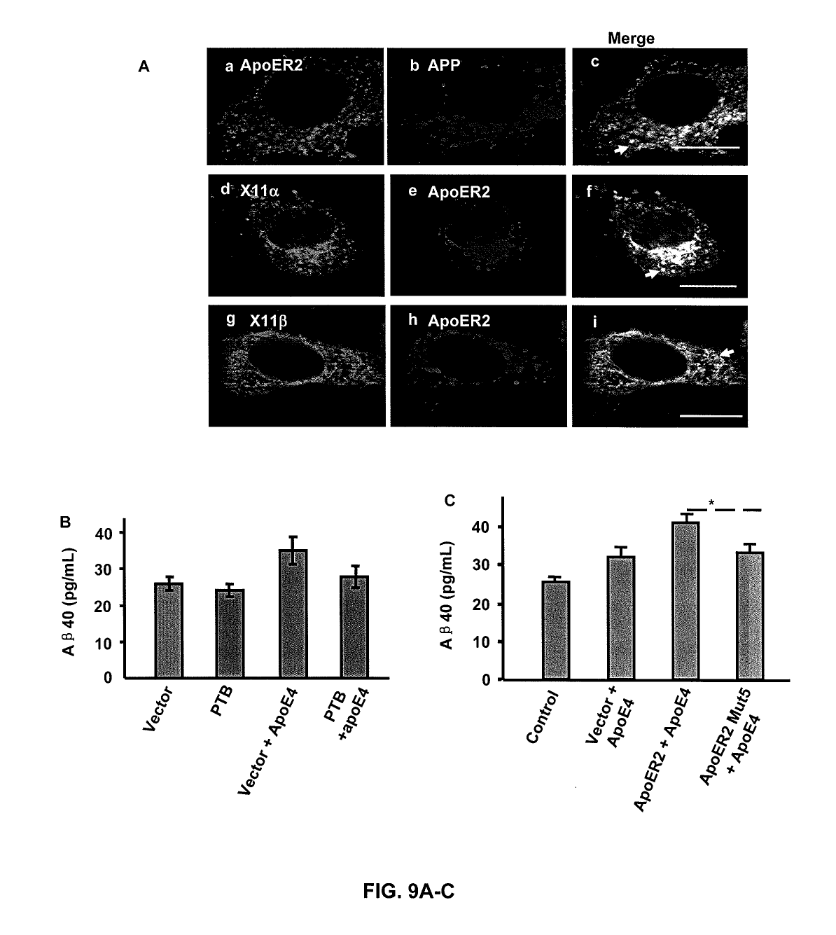

X11α/β involved in ApoE stimulated Aβ production. Since X11α/β binds to both ApoER2 and APP and is required for the formation of a complex containing three (FIGS. 7A-E & 8A-D), the inventor hypothesizes that it is involved in facilitating endocytosis of these proteins leading to ApoE induced Aβ production. This hypothesis is supported by the intracellular co-localization of APP with ApoER2 (FIGS. 9A-C) and ApoER2 with X11α/β (FIGS. 9D-I), especially in subcellular compartments consistent with endosomes (FIGS. 9A-I arrows). However, it is also known (Borg et al., 1998; Sastre et al., 1998) and the inventor has confirmed (results not shown) that the expression of X11α/β inhibits Aβ production. Such a response seems to contradict with the hypothesis. Therefore, the inventor sought evidence on the involvement of endogenous X11α/β in the ApoE induced Aβ increase from N2a cells. First, transfected PTB domain from X11α was used as dominant-negative inhibitor for the function of endogenous X11α/β. The inventor observed that ApoE4-induced Aβ increase (FIG. 9B, comparing columns 1 & 3) was largely abolished by PTB expression (FIG. 9B, column 4). Second, the expression of mutant ApoER2 with a deletion of the X11α/β-binding motif (mutant 5 in FIG. 4C) also greatly reduced Aβ increase induced by either ApoE4 or VLDL (FIG. 9C). These observations suggest that X11α/β binding at least to ApoER2 plays a role in ApoE induced endocytosis leading to the production of Aβ.

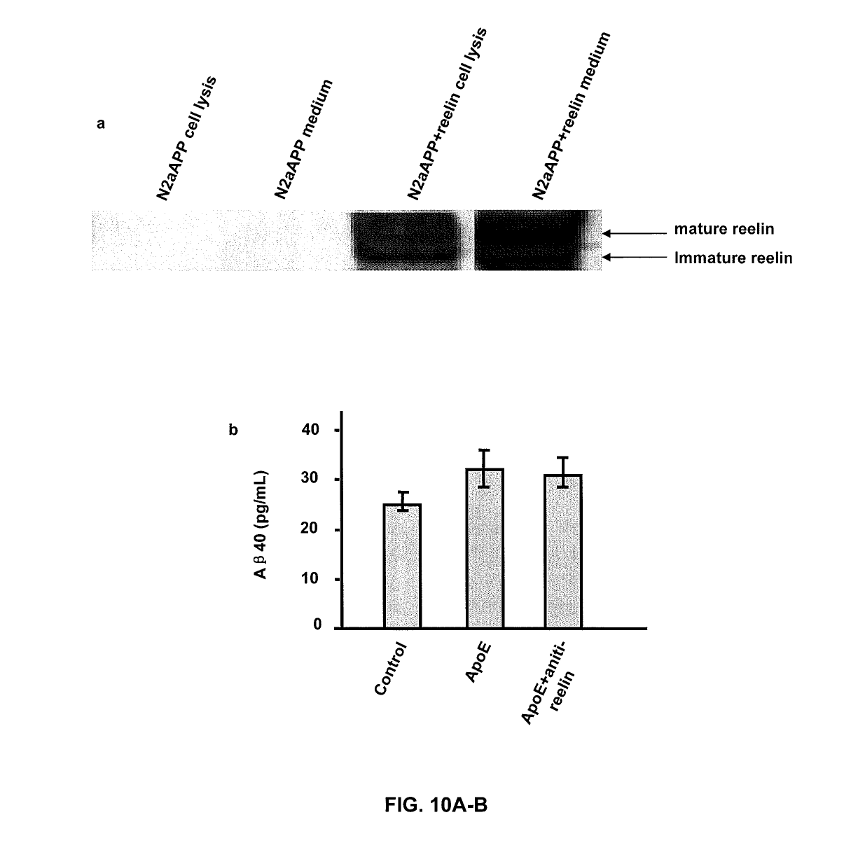

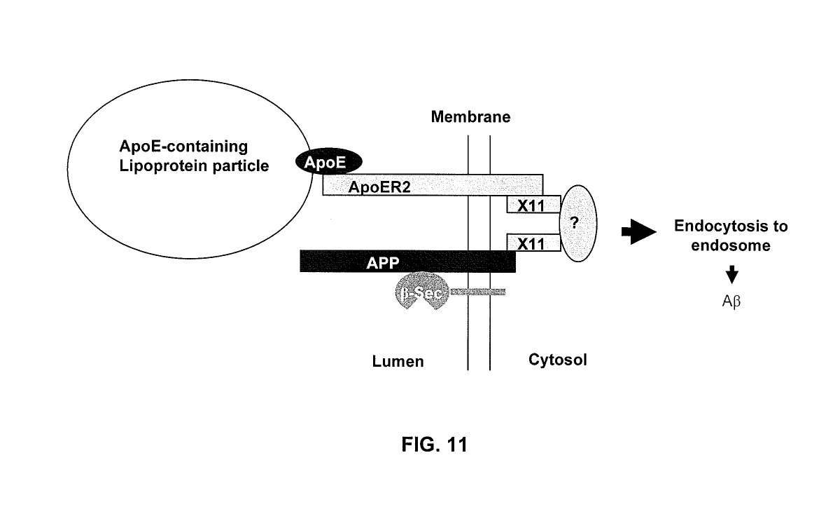

ApoER2-mediated Aβ increase by ApoE is independent of Reelin. ApoER2 is also a receptor for the neuronal signaling protein Reelin (D'Arcangelo et al., 1999; Hiesberger et al., 1999), which is known to reduce cellular Aβ production (Hoe et al., 2006). It is of interest then to determine whether ApoER2 mediated Aβ increase by ApoE is independent of Reelin-ApoER2 interaction. Western blots of N2a-APPsw culture medium or cell lysate concentrated from cell culture in T25 flasks did not detect the presence of Reelin (FIG. 10A, two left lanes) while the Reelin bands were clearly visible in cells transfected with a reelin expression vector (FIG. 10A, two right lanes). Moreover, the addition of anti-Reelin antibody to the culture mediate did not significantly alter the ApoE stimulated AD increase in N2a-APPsw cells (FIG. 10B). These observations indicate that ApoER2-mediated Aβ increase by ApoE is independent of Reelin-ApoER2 interaction.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. References

The following references, to the extent that they provide exemplary procedural or other details supplementary to those set forth herein, are specifically incorporated herein by reference.
U.S. Pat. No. 4,196,265
U.S. Pat. No. 4,415,723
U.S. Pat. No. 4,458,066
U.S. Pat. No. 4,816,567
U.S. Pat. No. 4,946,778
U.S. Pat. No. 5,223,424
U.S. Pat. No. 5,354,855
U.S. Pat. No. 5,440,013
U.S. Pat. No. 5,446,128
U.S. Pat. No. 5,475,085
U.S. Pat. No. 5,618,914
U.S. Pat. No. 5,626,850
U.S. Pat. No. 5,670,155
U.S. Pat. No. 5,672,681
U.S. Pat. No. 5,674,976
U.S. Pat. No. 5,710,245
U.S. Pat. No. 5,795,715
U.S. Pat. No. 5,840,833
U.S. Pat. No. 5,859,184
U.S. Pat. No. 5,888,773
U.S. Pat. No. 5,889,136
U.S. Pat. No. 5,929,237
U.S. Pat. No. 5,994,136
U.S. Pat. No. 6,165,782
U.S. Pat. No. 6,277,633
U.S. Pat. No. 6,319,703
U.S. Pat. No. 6,344,445
U.S. Pat. No. 6,428,953
U.S. Pat. No. 6,521,457
U.S. Pat. No. 6,924,144
U.S. Patent Pub. 20030147966
U.S. Patent Pub. 20030223938
U.S. Patent Pub. 20050143336
U.S. Provisional Application Ser. No. 60/661,680
Baichwal and Sugden, In: Gene Transfer, Kucherlapati (Ed.), NY, Plenum Press, 117-148, 1986.
Barany and Merrifield, In: The Peptides, Gross and Meienhofer (Eds.), Academic Press, NY, 1-284, 1979.
Beffert et al., J. Lipid Res., 45(3):403-9, 2004.
Borg et al. J. Neurosci., 19:1307-1316, 1999.
Borg et al., J. Biol. Chem., 273:14761-14766, 1998.
Bosher and Labouesse, Nat. Cell. Biol., 2:E31-E36, 2000.
Caplen et al., Gene, 252(1-2):95-105, 2000.
Clackson et al., Nature 352: 624-628, 1991.
Cook et al., Cell, 27:487-496, 1981.
D'Arcangelo et al., Neuron 24, 471-479, 1999.
Elbashir et al., Nature, 411(6836):494-498, 2001.
Fire et al., Nature, 391(6669):806-811, 1998.
Forster and Symons, Cell, 49(2):211-220, 1987.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gotthardt et al., J. Biol. Chem. 275:25616-25624, 2000.
Grishok et al., Science, 287:2494-2497, 2000.
Harlow and Lane, In: Antibodies: A Laboratory Manual, Cold Spring Harbor Laboratory, Cold Spring Harbor, N.Y., 346-348, 1988.
Hase et al., J. Neurochem., 81:1223-1232, 2002.
He et al., Biochemistry, 42:12174-12180, 2003.
He et al., J Biol. Chem., 280 (12):11696-703, 2005.
Hiesberger et al., Neuron., 24:481-489, 1999.
Hoe et al., J. Biol. Chem., 281:35176-35185, 2006.
Johannesson et al., J. Med. Chem., 42(22):4524-4537, 1999.
Johnson et al., In: Biotechnology And Pharmacy, Pezzuto et al. (Eds.), Chapman and Hall, NY, 1993.
Joyce, Nature, 338:217-244, 1989.
Kang et al., Nature, 325:733-736, 1987.
Ketting et al., Cell, 99(2):133-141, 1999.
Kim and Cook, Proc. Natl. Acad. Sci. USA, 84(24):8788-8792, 1987.
Kim et al., Neuron, 29(3):571-81, 2001.
King & Turner, Exp Neurol., 185(2):208-19, 2005.
Kohler et al., Nature 256: 495, 1975.
Lin and Avery, Nature, 402:128-129, 1999.
Lin et al., Proc. Nat'l Acad. Sci., 97:1456-1460, 2000.
Manchekar et al., J. Biol. Chem., 279:39757-39766, 2004.
Marks et al., J. Mol. Biol. 222: 581-597, 1991.
Merrifield, Science, 232(4748):341-347, 1986.
Michel and Westhof, J. Mol. Biol., 216:585-610, 1990.
Montgomery et al., Proc. Natl. Acad. Sci. USA, 95:15502-15507, 1998.
Motoi et al., Neurosci Lett., 368(2):144-7, 2004.
Mucke et al., J. Neuroscience 20:4050-4058, 2000.
Nicolas and Rubenstein, In: Vectors: A survey of molecular cloning vectors and their uses, Rodriguez and Denhardt, eds., Stoneham: Butterworth, pp. 494-513, 1988.
Nicolau et al., Methods Enzymol., 149:157-176, 1987.
PCT Appln. WO 00/44914
PCT Appln. WO 01/36646
PCT Appln. WO 01/68836
PCT Appln. WO 98/07408
PCT Appln. WO 99/32619
Petit-Turcotte et al., Neurobiol Aging, 26(2):195-206, 2005.
Price & Sisodia, Ann. Rev. Neurosci., 21:479-505, 1998.
Reinhold-Hurek and Shub, Nature, 357:173-176, 1992.
Remington's Pharmaceutical Sciences, 18th Ed. Mack Printing Company, pp. 1289-1329, 1990.
Riddell et al., J. Lipid Res. 40:1925-1930, 1999.
Ridgeway, In: Vectors: A Survey of Molecular Cloning Vectors and Their Uses, Rodriguez et al. (Eds.), Stoneham: Butterworth, 467-492, 1988.
Sarver et al., Science, 247:1222-1225, 1990.
Sastre et al., J. Biol. Chem., 273:22351-22357, 1998.
Scanlon et al., Proc. Natl. Acad. Sci. USA, 88:10591-10595, 1991.
Selkoe, D. J., Physiol. Rev., 81:741-766, 2001.
Sharp and Zamore, Science, 287:2431-2433, 2000.
Sharp, Genes Dev., 13:139-141, 1999.
Smyth-Templeton et al., DNA Cell Biol., 21(12):857-867, 1997.
Stewart and Young, In: Solid Phase Peptide Synthesis, 2d. ed., Pierce Chemical Co., 1984.
Stockinger et al., J. Biol. Chem. 275:25625-25632, 2000.
Sun & Soutar, Eur. J. Biochem. 262:230-239, 1999.
Tabara et al., Cell, 99(2):123-132, 1999.
Tam et al., J. Am. Chem. Soc., 105:6442, 1983.
Temin, In: Gene Transfer, Kucherlapati (Ed.), NY, Plenum Press, 149-188, 1986.
Trommsdorff et al., Cell 97:689-701, 1999.
Vita et al., Biopolymers 47:93-100, 1998.
Weisshoff et al., Eur. J. Biochem., 259(3):776-788, 1999.
Wincott et al., Nucleic Acids Res., 23(14):2677-2684, 1995.
Wong et al., Gene, 10:87-94, 1980.

What is claimed is:

1. A method of reducing Aβ production in a neuronal cell expressing an ApoER2 receptor comprising providing to said cell an agent that inhibits the binding of ApoE to ApoER2.

2. The method of claim 1, wherein said agent preferentially inhibits the interaction of ApoE4 binding to ApoER2.

3. The method of claim 1, wherein said agent is a soluble form of ApoER2 or an ApoER2 peptide.

4. The method of claim 1, wherein said agent is an ApoE peptide.

5. The method of claim 4, wherein said ApoE peptide is an ApoE4 peptide.

6. The method of claim 5, wherein said ApoE4 peptide comprises position 112 of the native ApoE4 protein.

7. The method of claim 1, wherein said agent is an antibody or antibody fragment that binds to ApoER2 or ApoE4.

8. The method of claim 7, wherein said antibody is a single chain antibody or a humanized antibody.

9. The method of claim 1, wherein said agent reduces the expression of ApoER2 in said neuronal cell.

10. The method of claim 9, wherein said agent is a small molecule, an ApoER2 antisense molecule, an ApoER2 siRNA, or an ApoER2 ribozyme.

11. The method of claim 1, wherein said agent reduces the expression of ApoE4 in a cell expressing ApoE4.

12. The method of claim 11, wherein said agent is a small molecule having binding affinity to ApoER2 or ApoE isomorphic forms, an ApoE4 antisense molecule, an ApoE4 siRNA, or an ApoE4 ribozyme.

13. The method of claim 1, wherein said agent is delivered in a lipid vehicle.

14. The method of claim 13, wherein said lipid vehicle is a liposome.

15. The method of claim 1, wherein providing comprises delivery to said neuronal cell or said cell expressing ApoE4 of an expression construct encoding said agent under the control of a promoter.

16. The method of claim 15, wherein said expression construct is a viral expression construct.

17. The method of claim 16, wherein said viral expression construct is neurotrophic virus.

18. The method of claim 17, wherein said neurotrophic virus is a retrovirus, a lentivirus, or a herpesvirus.

19. The method of claim 1, wherein said neuronal cell is a human neuronal cell.

20. The method of claim 19, wherein said human neuronal cell is in a living subject.

21. The method of claim 20, wherein said living subject suffers from Alzheimer's Disease.

22. The method of claim 20, wherein said living subject does not suffer from Alzheimer's Disease.

23. The method of claim 20, wherein said living subject has pre-existing Aβ plaques.

24. A method of inhibiting Aβ plaque formation in neurons of a subject comprising providing to subject an agent that inhibits the binding of ApoE to ApoER2.

25. A method of blocking the progression of one or more symptoms of Alzheimer's Disease in a subject comprising providing to subject an agent that inhibits the binding of ApoE to ApoER2.

26. A method of delaying the onset of Alzheimer's Disease in a subject comprising providing to subject an agent that inhibits the binding of ApoE to ApoER2.

27. A method of reducing Aβ production in a neuronal cell expressing an ApoER2 receptor comprising providing to said cell an agent that inhibits the binding of X11α/β to ApoER2.

28. The method of claim 27, wherein said agent is a dominant-negative form of X11α/β.

29. The method of claim 28, wherein said dominant-negative form of X11α/β is PTB domain peptide.

30. The method of claim 29, wherein PTB domain peptide comprises the sequence of SEQ ID NO:9, SEQ ID NO:10 or SEQ ID NO:12 or an inhibitory fragment thereof.

31. The method of claim 27, wherein said agent is a an antibody or antibody fragment that binds to ApoER2 or X11α/β.

32. The method of claim 31, wherein said antibody is a single chain antibody or a humanized antibody.

33. The method of claim 27, wherein said agent is delivered in a lipid vehicle.

34. The method of claim 33, wherein said lipid vehicle is a liposome.

35. The method of claim 27, wherein said neuronal cell is a human neuronal cell.

36. The method of claim 35, wherein said human neuronal cell is in a living subject.

37. The method of claim 36, wherein said living subject suffers from Alzheimer's Disease.

38. The method of claim 36, wherein said living subject does not suffer from Alzheimer's Disease.

39. The method of claim 36, wherein said living subject has pre-existing Aβ plaques.

40. The method of claim 27, wherein said agent is a peptidomimetic of X11α/β ApoE.

41. A method of inhibiting Aβ plaque formation in neurons of a subject comprising providing to said cell an agent that inhibits the binding of X11α/β to ApoER2.

42. A method of blocking the progression of one or more symptoms of Alzheimer's Disease in a subject comprising providing to said cell an agent that inhibits the binding of X11α/β to ApoER2.

43. A method of delaying the onset of Alzheimer's Disease in a subject comprising providing to said cell an agent that inhibits the binding of X11α/β to ApoER2.

44. A dominant negative X11α/β.

45. The dominant negative X11α/β of claim 44, wherein said dominant negative X11α/β lacks the PDZ domain.

46. A peptide comprising a phosphotyrosine/tyrosine binding (PTB) domain of X11α/β, said peptide being 10 to 50 residues in length.

47. The peptide of claim 46, comprising residues 413 to 421 of SEQ ID NO:8.

48. An antibody that binds to the phosphotyrosine/tyrosine binding (PTB) domain of X11α/β.

49. The antibody of claim 48, wherein said antibody is a single chain antibody or a humanized antibody.

50. An antisera, antibodies of which bind to the phosphotyrosine/tyrosine binding (PTB) domain of X11α/β.


SEQUENCE LISTING (12 sequences)

SEQ ID NO: 1 - protein, 317 aa, Homo sapiens [sequence omitted]

SEQ ID NO: 2 - protein, 963 aa, Homo sapiens
MGLPEPGPLRLLALLLLLLLLLLLRLQHLAAAAADPLLGGQGPAKECEKDQFQCRNERCIPSVWRCDEDDDCLDHSDEDDCPKKTCADSDFTCDNGHCIHERWKCDGEEECPDGSDESEATCTKQVCPAEKLSCGPTSHKCVPASWRCDGEKDCEGGADEAGCATLCAPHEFQCGNRSCLAAVFVCDGDDDCGDGSDERGCADPACGPREFRCGGDGGGACIPERWVCDRQFDCEDRSDEAAELCGRPGPGATSAPAACATVSQFACRSGECVHLGWRCDGDRDCKDKSDEADCPLGTCRGDEFQCGDGTCVLAIKHCNQEQDCPDGSDEAGCLQGLNECLHNNGGCSHICTDLKIGFECTCPAGFQLLDQKTCGDIDECKDPDACSQICVNYKGYFKCECYPGYEMDLLTKNCKAAGGKSPSLIFTNRYEVRRIDLVKRNYSRLIPMLKNVVALDVEVATNRIYWCDLSYRKIYSAYMDKASDPKEQEVLIDEQLHSPEGLAVDWVHKHIYWTDSGNKTISVATVDGGRRRTLFSRNLSEPRAIAVDPLRGFMYWSDWGDQAKIEKSGLNGVDRQTLVSDNIEWPNGITLDLLSQRLYWVDSKLHQLSSIDFSGGNRKTLISSTDFLSHPFGIAVFEDKVFWTDLENEAIFSANRLNGLEISILAENLNNPHDIVIFHELKQPRAPDACELSVQPNGGCEYLCLPAPQISSHSPKYTCACPDTMWLGPDMKRCYRAPQSTSTTTLASTMTRTVPATTRAPGTTVHRSTYQNHSTETPSLTAAVPSSVSVPRAPSISPSTLSPATSNHSQHYANEDSKMGSTVTAAVIGIIVPIVVIALLCMSGYLIWRNWKRKNTKSMNFDNPVYRKTTEEEDEDELHIGRTAQIGHVYPAAISSFDRPLWAEPCLGETREPEDPAPALKELFVLPGEPRSQLHQLPKNPLSELPVVKSKRVALSLEDDGLP

SEQ ID NO: 3 - protein, 860 aa, Homo sapiens [sequence omitted]

SEQ ID NO: 4 - protein, 873 aa, Homo sapiens
MGTSALWALWLLLALCWAPRESGATGTGRKAKCEPSQFQCTNGRCITLLWKCDGDEDCVDGSDEKNCVKKTCAESDFVCNNGQCVPSRWKCDGDPDCEDGSDESPEQCHMRTCRIHEISCGAHSTQCIPVSWRCDGENDCDSGEDEENCGNITCSPDEFTCSSGRCISRNFVCNGQDDCSDGSDELDCAPPTCGAHEFQCSTSSCIPISWVCDDDADCSDQSDESLEQCGRQPVIHTKCPASEIQCGSGECIHKKWRCDGDPDCKDGSDEVNCPSRTCRPDQFECEDGSCIHGSRQCNGIRDCVDGSDEVNCKNVNQCLGPGKFKCRSGECIDISKVCNQEQDCRDWSDEPLKECHINECLVNNGGCSHICKDLVIGYECDCAAGFELIDRKTCGDIDECQNPGICSQICINLKGGYKCECSRGYQMDLATGVCKAVGKEPSLIFTNRRDIRKIGLERKEYIQLVEQLRNTVALDADIAAQKLFWADLSQKAIFSASIDDKVGRHVKMIDNVYNPAAIAVDWVYKTIYWTDAASKTISVATLDGTKRKFLFNSDLREPASIAVDPLSGFVYWSDWGEPAKIEKAGMNGFDRRPLVTADIQWPNGITLDLIKSRLYWLDSKLHMLSSVDLNGQDRRIVLKSLEFLAHPLALTIFEDRVYWIDGENEAVYGANKFTGSELATLVNNLNDAQDIIVYHELVQPSGKNWCEEDMENGGCEYLCLPAPQINDHSPKYTCSCPSGYNVEENGRDCQSTATTVTYSETKDTNTTEISATSGLVPGGINVTTAVSEVSVPPKGTSAAWAILPLLLLVMAAVGGYLMWRNWQHKNMKSMNFDNPVYLKTTEEDLSIDIGRHSASVGHTYPAISVVSTDDDLA

SEQ ID NO: 5 - protein, 4 aa, Artificial Sequence
  Description of Artificial Sequence  Synthetic Peptide
NPTY

SEQ ID NO: 6 - protein, 4 aa, Artificial Sequence
  Description of Artificial Sequence  Synthetic Peptide
  MOD_RES (2)..(3): x = any natural occuring amino acid
PXXP

SEQ ID NO: 7 - protein, 6 aa, Artificial Sequence
  Description of Artificial Sequence  Synthetic Peptide
YDRPLW

SEQ ID NO: 8 - protein, 837 aa, Homo sapiens
MNHLEGSAEVEVTDEAAGGEVNESVEADLEHPEVEEEQQQPPQQQHYVGRHQRGRALEDLRAQLGQEEEERGECLARSASTESGFHNHTDTAEGDVIAAARDGYDAERAQDPEDESAYAVQYRPEAEEYTEQAEAEHAEATHRRALPNHLHFHSLEHEEAMNAAYSGYVYTHRLFHRGEDEPYSEPYADYGGLQEHVYEEIGDAPELDARDGLRLYEQERDEAAAYRQEALGARLHHYDERSDGESDSPEKEAEFAPYPRMDSYEQEEDIDQIVAEVKQSMSSQSLDKAAEDMPEAEQDLERPPTPAGGRPDSPGLQAPAGQQRAVGPAGGGEAGQRYSKEKRDAISLAIKDIKEAIEEVKTRTIRSPYTPDEPKEPIWVMRQDISPTRDCDDQRPMDGDSPSPGSSSPLGAESSSTSLHPSDPVEASTNKESRKSLASFPTYVEVPGPCDPEDLIDGIIFAANYLGSTQLLSDKTPSKNVRMMQAQEAVSRIKMAQKLAKSRKKAPEGESQPMTEVDLFISTQRIKVLNADTQETMMDHPLRTISYIADIGNIVVLMARRRMPRSNSQENVEASHPSQDGKRQYKMICHVFESEDAQLIAQSIGQAFSVAYQEFLRANGINPEDLSQKEYSDLLNTQDMYNDDLIHFSKSENCKDVFIEKQKGEILGVVIVESGWGSILPTVIIANMMHGGPAEKSGKLNIGDQIMSINGTSLVGLPLSTCQSIIKGLKNQSRVKLNIVRCPPVTTVLIRRPDLRYQLGFSVQNGIICSLMRGGIAERGGVRVGHRIIEINGQSVVATPHEKIVHILSNAVGEIHMKTMPAAMYRLLTAQEQPVYI

SEQ ID NO: 9 - protein, 171 aa, Homo sapiens
GQQRAVGPAGGGEAGQRYSKEKRDAISLAIKDIKEAIEEVKTRTIRSPYTPDEPKEPIWVMRQDISPTRDCDDQRPMDGDSPSPGSSSPLGAESSSTSLHPSDPVEASTNKESRKSLASFPTYVEVPGPCDPEDLIDGIIFAANYLGSTQLLSDKTPSKNVRMMQAQEAVS

SEQ ID NO: 10 - protein, 187 aa, Homo sapiens
DGIIFAANYLGSTQLLSDKTPSKNVRMMQAQEAVSRIKMAQKLAKSRKKAPEGESQPMTEVDLFILTQRIKVLNADTQETMMDHPLRTISYIADIGNIVVLMARRRIPRSNSQENVEASHPSQDGKRQYKMICHVFESEDAQLIAQSIGQAFSVAYQEFLRANGINPEDLSQKEYSDLLNTQDMYND

SEQ ID NO: 11 - protein, 749 aa, Homo sapiens
MAHRKLESVGSGMLDHRVRPGPVPHSQEPESEDMELPLEGYVPEGLELAALRPESPAPEEQECHNHSPDGDSSSDYVNNTSEEEDYDEGLPEEEEGITYYIRYCPEDDSYLEGMDCNGEEYLAHSAHPVDTDECQEAVEEWTDSAGPHPHGHEAEGSQDYPDGQLPIPEDEPSVLEAHDQEEDGHYCASKEGYQDYYPEEANGNTGASPYRLRRGDGDLEDQEEDIDQIVAEIKMSLSMTSITSASEASPEHGPEPGPEDSVEACPPIKASCSPSRHEARPKSLNLLPEAKHPGDPQRGFKPKTRTPEERLKWPHEQVCNGLEQPRKQQRSDLNGPVDNNNIPETKKVASFPSFVAVPGPCEPEDLIDGIIFAANYLGSTQLLSERNPSKNIRMMQAQEAVSRVKRMQKAAKIKKKANSEGDAQTLTEVDLFISTQRIKVLNADTQETMMDHALRTISYIADIGNIVVLMARRRMPRSASQDCIETTPGAQEGKKQYKMICHVFESEDAQLIAQSIGQAFSVAYQEFLRANGINPEDLSQKEYSDIINTQEMYNDDLIHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRPDLKYQLGFSVQNGIICSLMRGGIAERGGVRVGHRIIEINGQSVVATAHEKIVQALSNSVGEIHMKTMPAAMFRLLTGQETPLYI

SEQ ID NO: 12 - protein, 165 aa, Homo sapiens
GIIFAANYLGSTQLLSERNPSKNIRMMQAQEAVSRVKRMQKAAKIKKKANSEGDAQTLTEVDLFISTQRIKVLNADTQETMMDHALRTISYIADIGNIVVLMARRRMPRSASQDCIETTPGAQEGKKQYKMICHVFESEDAQLIAQSIGQAFSVAYQEFLRANGI